(19) United States
(12) Patent Application Publication
(10) Pub. No.: US 2023/0026277 A1
(43) Pub. Date: Jan. 26, 2023

(54) COMPOSITION FOR PREVENTION, ALLEVIATION, OR TREATMENT OF RESPIRATORY DISEASE

(71) Applicant: MICROX CO., LTD., Seoul (KR)

(72) Inventors: Hern Ku LEE, Jeonju-si, Jeollabuk-do (KR); Myung Kwan HAN, Jeonju-si, Jeollabuk-do (KR); Ki Bum HONG, Daegu (KR); Ji Hoon LEE, Seoul (KR); Ji Hoon YU, Hwaseong-si, Gyeonggi-do (KR); Jusuk LEE, Daegu (KR); Shun Young IM, Gwangju (KR); Hwa Ryung SONG, Jeonju-si, Jeollabuk-do (KR)

(73) Assignee: MICROX CO., LTD., Seoul (KR)

(21) Appl. No.: 17/756,534

(22) PCT Filed: Nov. 24, 2020
(86) PCT No.: PCT/KR2020/016654

§ 371 Date: May 26, 2022

(30) Foreign Application Priority Data
Nov. 27, 2019 (KR) 10-2019-0154651

(51) Int. Cl. A61K 31/223; A61P 11/00; A61K 31/198; A61K 31/216; A23L 33/00; A23L 33/10

(52) U.S. Cl. CPC A61K 31/223; A61P 11/00; A61K 31/198; A61K 31/216; A23L 33/40; A23L 33/10; A23V 2002/00

(57) ABSTRACT

Provided is a novel compound and a use thereof. A novel compound according to the presently claimed subject matter induces the activation of MKP-1 protein to block the cellular signaling pathway proceeding in the order of p38/CK2α/NF-κB, thereby very effectively inhibiting an inflammatory response occurring in a respiratory disease. Therefore, a respiratory disease can be prevented, alleviated, or treated by orally administering the novel compound according to the presently claimed subject matter.

### TECHNICAL FIELD

The present invention relates to a composition for preventing, alleviating, or treating a respiratory disease, which comprises a novel compound as an active ingredient.

This application claims priority to and the benefit of Korean Patent Application No. 10-2019-0154651, filed Nov. 27, 2019, the disclosure of which is incorporated herein by reference in its entirety.

### BACKGROUND ART

Asthma is one of chronic diseases, which has main symptoms such as bronchial hyperresponsiveness caused by allergic inflammation in the airway, and dyspnea accompanied by coughing caused by airway smooth muscle contraction. The disease often causes the spasms of the bronchial smooth muscle system, affecting both upper and lower airways. There are several types of asthma depending on the seventy of symptoms thereof. For example, mild asthma is defined as a symptom such as dyspnea or coughing, which may be absent. Moderate asthma is defined as wheezing and dyspnea, in which coughing and phlegm may or may not be present, but generally interferes with daily activities or sleep. Finally, severe asthma is characterized by incapacity due to dyspnea, and afflicted patients usually cannot eat or sleep normally, suffer from anxiety, and show symptoms of exhaustion. There are approximately 300 million patients suffering from asthma having such symptoms worldwide, and the number of deaths caused by asthma is 25,000 every year.

Meanwhile, chronic obstructive pulmonary disease (COPD) is a group of pulmonary diseases in which airways are narrowed. This restricts the flow of air into and out of the lungs, resulting in breathing difficulties. Unlike asthma which is a part of respiratory diseases, the airflow restriction is poorly reversible, and generally becomes progressively worse over time. COPD has main symptoms such as pulmonary emphysema and chronic obstructive bronchitis. In the pulmonary emphysema, walls between multiple air sacs are damaged so that they droop due to their shape deformation, and such damage induces damage between successive air sacs. Also, in the chronic obstructive bronchitis, inflammation is caused by continuous irritation in the inner layers of airways. As a result, the inner layers of airways become thick, and thus thick mucus is formed in the airways, which makes it difficult for patients to breathe.

The chronic bronchitis and the pulmonary emphysema may be most commonly caused by smoking, and approximately 90% of patients suffering from COPD currently smoke or have a history of smoking. Approximately 50% of smokers develop chronic bronchitis, but only 15% of the smokers develop irreversible airflow obstruction. However, it is known that the onset of COPD is not confined to humans only, and other mammals, for example, horses, also suffer from COPD.

COPD is an important cause of death and disability. It is the fourth leading cause of death in the United States and Europe, and also has a very high incidence rate in Asia. The guidelines for treatment recommend the implementation of early detection and smoking cessation programs to aid in reducing the morbidity and mortality from the disease. However, COPD has limitations in that it is not easily found at an early stage, and there are currently no drugs capable of inducing a cure at an advanced stage of the disease.

In the case of asthma, inhaled steroids and β2-agonists have been widely used depending on the severity of symptoms, but the asthma is not controlled in approximately 20% of the patients, and in 3 to 5% of the patients, high-dose steroids and β2-agonists do not cure at all. In particular, such inhaled therapeutic agents have side effects such as a distorted voice, oral infection by fungi, hormone imbalances, and the like because high concentration steroids are administered for a long period of time.

### DISCLOSURE

### Technical Problem

The present invention is designed to solve the problems of the related art, and thus it is an object of the present invention to provide a composition for preventing, alleviating or treating a respiratory disease, which comprises a novel compound according to the present invention as an active ingredient.

However, the technical objects of the present invention are not limited to the technical object as described above, and other objects of the present invention which are not stated herein will become more apparent to those of ordinary skill in the art by describing exemplary embodiments thereof in detail.

### Technical Solution

Therefore, the present invention provides a pharmaceutical composition for preventing or treating a respiratory disease, which comprises a compound selected from a compound represented by the following Formula 1, a pharmaceutically acceptable salt, a hydrate, and a solvate thereof as an active ingredient:

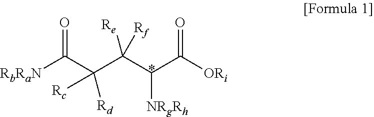

wherein:

Rₐ and R_{b} are each independently H or —C(═O)—Rⱼ;

R_{c} to R_{f} are each independently H or a C1-C6 alkyl group;

R_{g} and Rₕ are each independently H, a C1-C6 alkyl group, —C(═O)—Rₖ, or —C(═O)—O-L₂-Rₗ;

Rᵢ is H or a C1-C6 alkyl group;

Rⱼ and Rₖ are each independently a C1-C6 alkyl group;

Rₗ is a C1-C6 alkyl group, a C6-C12 aryl group, or a non-aromatic condensed polycyclic group having 5 to 20 nuclear atoms;

L₂ is a direct bond or a C1-C6 alkylene group;

the alkyl group of Rᵢ is substituted or unsubstituted with one or more C6-C12 aryl groups, which are the same or different from each other when substituted with a plurality of substituents; and

* represents a chiral center.

According to one embodiment of the present invention, the compound may be in an L form based on the chiral center represented by *, but the present invention is not limited thereto.

According to another embodiment of the present invention, the compound represented by Formula 1 may be represented by the following Formula 2, but the present invention is not limited thereto:

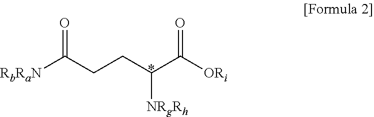

Rₐ and R_{b} are each independently H or —C(═O)—Rⱼ;

R_{g} and Rₕ are each independently H, a C1-C6 alkyl group, —C(═O)—Rₖ, or —C(═O)—O-L₂-Rₗ;

Rᵢ is H or a C1-C6 alkyl group;

Rⱼ and Rₖ are each independently a C1-C6 alkyl group;

Rₗ is a C1-C6 alkyl group, a C6-C12 aryl group, or a non-aromatic condensed polycyclic group having 5 to 20 nuclear atoms;

L₂ is a direct bond or a C1-C6 alkylene group;

the alkyl group of Rᵢ is substituted or unsubstituted with one or more C6-C12 aryl groups, which are the same or different from each other when substituted with a plurality of substituents; and

* represents a chiral center.

According to still another embodiment of the present invention, the compound may be one or more selected from the group consisting of the following compounds, but the present invention is not limited thereto:

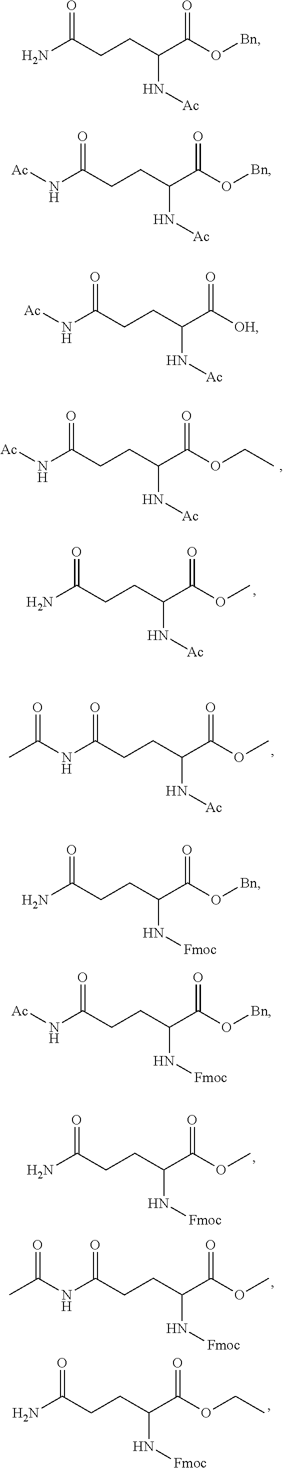

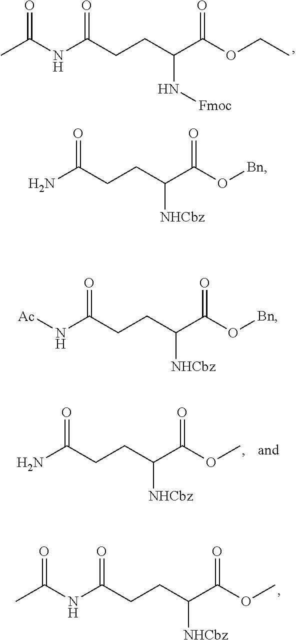

According to still another embodiment of the present invention, the respiratory disease may be one or more selected from the group consisting of a cold accompanied by coughing or phlegm, influenza, asthma, chronic obstructive pulmonary disease, bronchial adenoma, a solitary pulmonary nodule, pulmonary tuberculosis, empyema, a pulmonary abscess, and pulmonary histiocytosis, but the present invention is not limited thereto.

According to still another embodiment of the present invention, the chronic obstructive pulmonary disease may exhibit one or more symptoms of chronic bronchitis or pulmonary emphysema, but the present invention is not limited thereto.

Also, the present invention provides a food composition for preventing or alleviating a respiratory disease, which comprises the compound represented by the above Formula 1 or a pharmaceutically acceptable salt thereof as an active ingredient.

In addition, the present invention provides a use of a compound selected from the compound represented by the above Formula 1, a pharmaceutically acceptable salt, a hydrate, and a solvate thereof for producing a medicament for treatment of a respiratory disease.

Additionally, the present invention provides a method of preventing or treating a respiratory disease, which comprises administering a pharmaceutical composition comprising a compound selected from the compound represented by the above Formula 1, a pharmaceutically acceptable salt, a hydrate, and a solvate thereof to a subject.

Further, the present invention provides a use of the pharmaceutical composition comprising a compound selected from the compound represented by the above Formula 1, a pharmaceutically acceptable salt, a hydrate, and a solvate thereof for preventing or treating a respiratory disease.

### Advantageous Effects

A novel compound according to the present invention induces the activation of MKP-1 protein to block a cellular signaling pathway proceeding in the order of p38/CK2α/NF-κB, thereby very effectively inhibiting an inflammatory response occurring in a respiratory disease. Therefore, a respiratory disease can be prevented, alleviated, or treated by orally administering the novel compound according to the present invention.

### DESCRIPTION OF DRAWINGS

FIG. 1 is a schematic diagram of an experiment protocol for establishing an asthma-induced animal model according to one embodiment of the present invention.

FIG. 2 is a schematic diagram of an experiment protocol for establishing a chronic obstructive pulmonary disease-induced animal model according to one embodiment of the present invention.

A and B of FIG. 3 show the results of measuring the number of immune cells present in the bronchus according to one embodiment of the present invention, as observed through Diff-Quick staining under a microscope.

A and B of FIG. 4 show the results of measuring expression levels of cytokines (IL-4, IL-5 and IL-13) present in the bronchus according to one embodiment of the present invention, as determined through ELISA.

A and B of FIG. 5 show the results of measuring a bronchial hypersensitivity reaction according to one embodiment of the present invention using a method in which the hypersensitivity reaction may be indicated as a pulmonary resistance (Rrs) value.

A and B of FIG. 6 show the results of determining a degree of inflammation confirmed in a lung tissue according to one embodiment of the present invention, as determined through lung tissue staining.

FIG. 7 shows the results of determining levels of expression and activation of an MKP-1 protein, a p38 protein, and a cPLA2 protein according to one embodiment of the present invention, as determined through Western blot analysis.

FIG. 8 shows the results of determining levels of expression and activation of an MKP-1 protein, a p38 protein, and a cPLA2 protein when treated with siRNA specific to the MKP-1 protein according to one embodiment of the present invention, as determined through Western blot analysis.

A and B of FIG. 9 show the results of determining the inhibition of CK2/NF-κB activation dependent on the MKP-1 protein according to one embodiment of the present invention, as determined through Western blot analysis and ELISA analysis.

FIG. 10 show the results of determining the hierarchical relationship between a CK2α protein and p38 according to one embodiment of the present invention, as determined through Western blot analysis.

A and B of FIG. 11 show the results of confirming an inhibitory effect on NF-κB activation by the inhibition of the CK2α protein according to one embodiment of the present invention, as determined through Western blot analysis and ELISA analysis.

FIG. 12 shows the results of measuring the number of immune cells present in the bronchus when treated with siRNA specific to the MKP-1 protein according to one embodiment of the present invention, as observed through Diff-Quick staining under a microscope.

FIG. 13 shows the results of measuring expression levels of cytokines (IL-4, IL-5, and IL-13) present in the bronchus when treated with siRNA specific to the MKP-1 protein according to one embodiment of the present invention, as determined through ELISA.

FIG. 14 shows the results of measuring a bronchial hypersensitivity reaction when treated with siRNA specific to the MKP-1 protein according to one embodiment of the present invention using a method in which the hypersensitivity reaction may be indicated as the Rrs value.

A and B of FIG. 15 show the results of determining a degree of inflammation confirmed in a lung tissue when treated with siRNA specific to the MKP-1 protein according to one embodiment of the present invention, as determined through lung tissue staining.

FIG. 16 is a schematic diagram showing a cellular signaling process associated with the inhibition of inflammation by the novel compound according to one embodiment of the present invention.

FIG. 17 shows the results of determining an expression level of elastin in mice with chronic obstructive pulmonary disease according to one embodiment of the present invention, as determined through Western blot analysis.

FIG. 18 shows the results of determining expression levels of caspase-3 and collagen proteins in the mice with chronic obstructive pulmonary disease according to one embodiment of the present invention, as determined through Western blot analysis.

FIG. 19 shows the results of measuring the number of immune cells present in the bronchus when treated with various novel compounds according to one embodiment of the present invention, as observed through Diff-Quick staining under a microscope.

FIG. 20 shows the results of measuring an expression level of a cytokine (IL-5) present in the bronchus when treated with the various novel compounds according to one embodiment of the present invention, as determined through ELISA.

FIG. 21 shows the results of measuring a bronchial hypersensitivity reaction when treated with the various novel compounds according to one embodiment of the present invention using a method in which the hypersensitivity reaction may be indicated as the Rrs value.

FIG. 22 shows the results of comparison of degrees of inhibition of inflammatory cells between an existing asthma therapeutic agent and the novel compound according to one embodiment of the present invention.

FIG. 23 shows the results of comparison of degrees of inhibition of the expression of a cytokine (IL-5) between the existing asthma therapeutic agent and the novel compound according to one embodiment of the present invention.

### BEST MODE

According to one embodiment of the present invention, there is provided a composition for preventing, alleviating or treating a respiratory disease.

The composition of the present invention comprises a compound selected from a compound represented by the following Formula 1, a pharmaceutically acceptable salt, a hydrate, and a solvate thereof as an active ingredient:

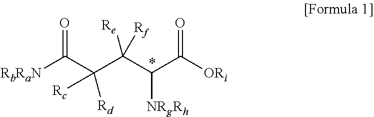

wherein:

Rₐ and R_{b} are each independently H or —C(═O)—Rⱼ;

R_{c} to R_{f} are each independently H or a C1-C6 alkyl group;

R_{g} and Rₕ are each independently H, a C1-C6 alkyl group, —C(═O)—Rₖ, or —C(═O)—O-L₂-Rₗ;

Rᵢ is H or a C1-C6 alkyl group;

Rⱼ and Rₖ are each independently a C1-C6 alkyl group;

Rₗ is a C1-C6 alkyl group, a C6-C12 aryl group, or a non-aromatic condensed polycyclic group having 5 to 20 nuclear atoms;

L₂ is a direct bond or a C1-C6 alkylene group;

the alkyl group of Rᵢ is substituted or unsubstituted with one or more C6-C12 aryl groups, which are the same or different from each other when substituted with a plurality of substituents; and

* represents a chiral center.

In the present invention, the term “alkyl group” may be linear or branched, and refers to a saturated monovalent hydrocarbon radical. Here, the alkyl may be unsubstituted or optionally substituted with one or more substituents described in the present invention. The alkyl group of the present invention may be an alkyl group having 1 to 6 carbon atoms, for example, a methyl group, an ethyl group, a propyl group, an isopropyl group, a butyl group, a t-butyl group, an n-butyl group, an isobutyl group, a sec-butyl group, a hexyl group, or the like, but the present invention is not limited thereto.

In the present invention, the term “alkylene group” is a form in which one hydrogen atom is removed from carbon atoms at both ends of a —CnH₂n-alkane, and may be linear or branched. Here, the alkylene group may be unsubstituted or optionally substituted with one or more substituents described in the present invention. The alkylene group of the present invention may be an alkylene group having 1 to 6 carbon atoms, for example, a methylene group, an ethylene group, a propylene group, an isopropylene group, a butylene group, a t-butylene group, an n-butylene group, an isobutylene group, a sec-butylene group, a hexylene group, or the like, but the present invention is not limited thereto.

Unless otherwise stated, in the present invention, the term “aryl group” refers to a mono- or poly-cyclic carbocyclic ring system having 6 to 12 carbon atoms, which includes one or more fused or non-fused aromatic rings. Here, examples of the aryl group may include a phenyl group, a halophenyl group, a tetrahydronaphthyl group, an indenyl group, an anthracenyl group, a benzyl group, a halobenzyl group, a naphthyl group, a biaryl group, or the like, but the present invention is not limited thereto.

In the present invention, the term “non-aromatic condensed polycyclic group” refers to a group that has two or more rings condensed with each other, contains 5 to 20 carbon atoms as ring forming atoms, and the entire molecule exhibits non-aromacity. Examples of the non-aromatic condensed polycyclic group may be fluorenyl, or the like, but the present invention is not limited thereto.

In the present invention, the term “substituted” means that one or more hydrogen atoms present in the alkyl group are substituted with a C1-C12 aryl group, but the present invention is not limited thereto.

In the present invention, the term “chiral center” refers to a carbon atom to which four other substituents are attached, and a compound which exhibits chirality may be identified when a molecule does not overlap an enantiomer thereof due to the structural difference based on the chiral center. Such chirality may be expressed as a D form or an L form, when a carboxyl group, a substituent, and an amino group is positioned in a clockwise or counterclockwise order around the carbon of the chiral center according to the CORN rule.

According to one preferred embodiment of the present invention, Rₐ and R_{b} may each be independently H or —C(═O)—Rⱼ.

According to one preferred embodiment of the present invention, R_{c} to R_{f} may each be independently H or a C1-C6 alkyl group. More preferably, all of R_{c} to R_{f} may be H.

According to one preferred embodiment of the present invention, any one of R_{g} and Rₕ may be H, and the other may be —C(═O)—Rₖ or —C(═O)—O-L₂-Rₗ.

According to one preferred embodiment of the present invention, Rᵢ may be a C1-C3 alkyl group, most preferably a methyl group or an ethyl group. The alkyl group of Rᵢ may be substituted with one or more C6-C12 aryl groups. More preferably, any one hydrogen atom of the alkyl group of Rᵢ may be substituted with a phenyl group.

According to one preferred embodiment of the present invention, Rⱼ and Rₖ may each be independently a C1-C3 alkyl group, more preferably a methyl group or an ethyl group.

According to one preferred embodiment of the present invention, Rₗ may be a C1-C6 alkyl group, a C6-C12 aryl group, or a non-aromatic condensed polycyclic group having 5 to 20 nuclear atoms, more preferably a phenyl group or a fluorene group.

According to one preferred embodiment of the present invention, L₂ may be a C1-C6 alkylene group, more preferably a C1-C3 alkylene group, and most preferably a methylene group or an ethylene group.

According to one preferred embodiment of the present invention, the symbol “*” represents a chiral center. In this case, the compound may be in an L form based on the chiral center represented by *.

The compound of the present invention may be a compound represented by the following Formula 2:

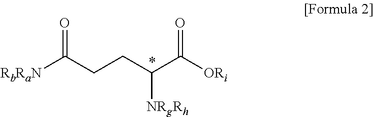

wherein:

each of Rₐ, R_{b}, and R_{g} to Rₗ, L₂, and * is as defined in Formula 1.

The compound of the present invention may be at least one selected from the group consisting of the following compounds:

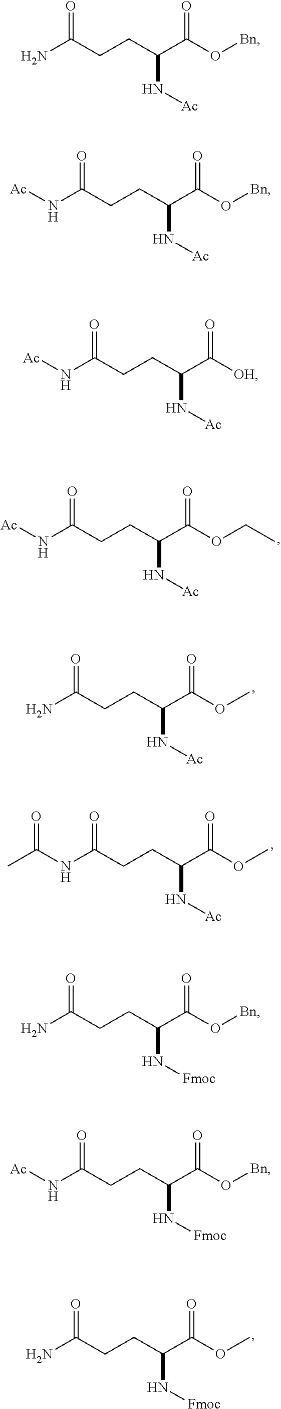

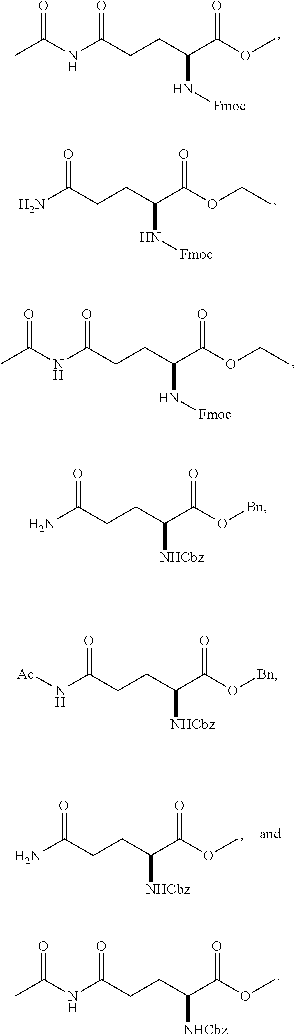

The respiratory disease of the present invention may be at least one selected from the group consisting of a cold accompanied by coughing or phlegm, influenza, asthma, chronic obstructive pulmonary disease, bronchial adenoma, a solitary pulmonary nodule, pulmonary tuberculosis, empyema, a pulmonary abscess, and pulmonary histiocytosis, but the present invention is not limited thereto.

The chronic obstructive pulmonary disease of the present invention may have at least any one symptom of chronic bronchitis or pulmonary emphysema, but the present invention is not limited thereto.

The composition of the present invention is not toxic even when the composition is orally administered to a target subject who has developed a respiratory disease. Therefore, the composition of the present invention may be used to very effectively treat a respiratory disease without any side effects such as a distorted voice caused by spraying a steroid drug.

The composition of the present invention may be used as a pharmaceutical composition or a food composition.

The present invention also provides a pharmaceutically acceptable salt of the compound represented by the above Formula 1 or 2. The pharmaceutically acceptable salt should have low toxicity to the human body, and should not adversely affect the biological activity and physicochemical properties of a parent compound. The pharmaceutically acceptable salt that may be used herein may include a pharmaceutically acceptable free acid, an acid addition salt of the base compound of Formula 1 or 2, and the like, but the present invention is not limited thereto.

Examples of suitable acids include hydrochloric acid, bromic acid, sulfuric acid, nitric acid, perchloric acid, fumaric acid, maleic acid, phosphoric acid, glycolic acid, lactic acid, salicylic acid, succinic acid, toluene-p-sulfonic acid, tartaric acid, acetic acid, citric acid, methanesulfonic acid, formic acid, benzoic acid, malonic acid, gluconic acid, naphthalene-2-sulfonic acid, benzenesulfonic acid, and the like. The acid addition salt may be prepared using a conventional method, for example, by dissolving a compound in an excessive amount of an acidic aqueous solution, and precipitating the salt using a water-miscible organic solvent such as methanol, ethanol, acetone, or acetonitrile. Also, the acid addition salt may be prepared by heating an equivalent molar amount of the compound and an acid or alcohol in water and evaporating or drying the resulting mixture, or prepared by filtering the precipitated salt by aspiration.

The salts derived from suitable bases may include alkali metals such as sodium, potassium, and the like; alkaline earth metals such as magnesium, and the like; ammonium; and the like, but the present invention is not limited thereto. The salts of the alkali metals or the alkaline earth metals may be, for example, obtained by dissolving a compound in an excessive amount of a solution of alkali metal hydroxide or alkaline earth metal hydroxide, filtering an insoluble salt of the compound, and evaporating and drying the filtrate. In this case, sodium, potassium or calcium salts are particularly pharmaceutically suitable as the metal salts. Also, silver salts corresponding to the metal salts may be obtained by allowing an alkali metal or alkaline earth metal salt to react with a proper silver salt (e.g., silver nitrate).

The salt of the present invention may be prepared using a conventional method. For example, the salt may be prepared by dissolving the described-above compound of Formula 1 or 2 in a solvent that is miscible with water, such as methanol, ethanol, acetone, 1,4-dioxane, and the like, adding a free acid or a free base thereto, and crystallizing the resulting mixture.

In addition to the compound of the present invention, the hydrate or solvate of the compound Formula 1 or 2 may also fall within the scope of the present invention.

A content of the compound in the composition of the present invention may be suitably adjusted depending on the symptom of a disease, a degree of progression of the disease, the conditions of a patient, and the like. For example, the content of the compound may be in a range of 0.0001 to 99.9% by weight, or 0.001 to 50% by weight, based on the total weight of the composition, but the present invention is not limited thereto. The content ratio is a value determined based on the dry mass from which the solvent is removed.

The pharmaceutical composition according to the present invention may further include a suitable carrier, excipient, and diluent typically used for preparing pharmaceutical compositions. For example, the excipient may include one or more selected from the group consisting of a diluent, a binder, a disintegrant, a lubricant, an adsorbent, a moisturizing agent, a film-coating material, and a controlled-release additive.

The pharmaceutical composition according to the present invention may be formulated into the form of a preparation for external use, such as a powder, granules, sustained-release granules, enteric granules, a liquid, an eye drop, an elixir, an emulsion, a suspension, a spirit, a troche, aromatic water, a limonade, a tablet, a sustained-release tablet, an enteric tablet, a sublingual tablet, a hard capsule, a soft capsule, a sustained-release capsule, an enteric capsule, a pill, a tincture, a soft extract, a dry extract, a fluid extract, an injection, a capsule, a perfusate, a plaster, a lotion, a paste, a spray, an inhalant, a patch, a sterile injectable solution, an aerosol, or the like, and used according to each of the conventional methods. The preparation for external use may have a formulation such as a cream, a gel, a patch, a spray, an ointment, a plaster, a lotion, a liniment, a paste, a cataplasma, or the like. For the purposes of the present invention, when fibrosis develops in the respiratory tract such as lungs, the active ingredient is preferably formulated for administration by inhalation so that the active ingredient can reach a target organ at a yield suitable for prevention or treatment.

The carrier, the excipient, and the diluent, which may be included in the pharmaceutical composition according to the present invention, may include lactose, dextrose, sucrose, an oligosaccharide, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, and mineral oil.

When formulated, the composition may be prepared using a commonly used diluent or excipient such as a filler, an extender, a binder, a wetting agent, a disintegrant, a surfactant, and the like.

Excipients such as corn starch, potato starch, wheat starch, milk sugar, white sugar, glucose, fructose, D-mannitol, precipitated calcium carbonate, synthetic aluminum silicate, calcium hydrogen phosphate, calcium sulfate, sodium chloride, sodium hydrogen carbonate, purified lanolin, microcrystalline cellulose, dextrin, sodium alginate, methyl cellulose, carboxymethyl cellulose sodium, kaolin, urea, colloidal silica gel, hydroxypropyl starch, hydroxypropyl methyl cellulose (HPMC), HPMC 1928, HPMC 2208, HPMC 2906, HPMC 2910, propylene glycol, casein, calcium lactate, Primojel, and the like; binders such as gelatin, gum arabic, ethanol, agar powder, cellulose acetate phthalate, carboxymethyl cellulose, carboxymethyl cellulose calcium, glucose, purified water, casein sodium, glycerin, stearic acid, carboxymethyl cellulose sodium, methyl cellulose sodium, methyl cellulose, microcrystalline cellulose, dextrin, hydroxycellulose, hydroxypropyl starch, hydroxymethyl cellulose, purified shellac, gelatinized starch, hydroxypropyl cellulose, hydroxypropyl methyl cellulose, polyvinyl alcohol, polyvinyl pyrrolidone, and the like may be used as the additives of the tablet, the powder, the granule, the capsule, the pill, and the troche according to the present invention. Also, disintegrants such as hydroxypropyl methyl cellulose, corn starch, agar powder, methyl cellulose, bentonite, hydroxypropyl starch, carboxymethyl cellulose sodium, sodium alginate, carboxymethyl cellulose calcium, calcium citrate, sodium lauryl sulfate, silicic acid anhydride, 1-hydroxypropyl cellulose, dextran, an ion exchange resin, polyvinyl acetate, formaldehyde-treated casein and gelatin, alginic acid, amylose, guar gum, sodium bicarbonate, polyvinyl pyrrolidone, calcium phosphate, gelatinized starch, gum arabic, amylopectin, pectin, sodium polyphosphate, ethyl cellulose, white sugar, magnesium aluminum silicate, a D-sorbitol solution, a light silicic acid anhydride, and the like; lubricants such as calcium stearate, magnesium stearate, stearic acid, a hydrogenated vegetable oil, talc, lycopodium powder, kaolin, Vaseline, sodium stearate, cacao butter, sodium salicylate, magnesium salicylate, polyethylene glycol 4000, 6000, liquid paraffin, a hydrogenated soybean oil (Lubri wax), aluminum stearate, zinc stearate, sodium lauryl sulfate, magnesium oxide, Macrogol, synthetic aluminum silicate, silicic acid anhydride, a higher fatty acid, a higher alcohol, silicone oil, paraffin oil, a polyethylene glycol fatty acid ester, starch, sodium chloride, sodium acetate, sodium oleate, D/L-leucine, a light silicic acid anhydride, and the like, may be used.

Water, dilute hydrochloric acid, dilute sulfuric acid, sodium citrate, sucrose monostearates, polyoxyethylene sorbitol fatty acid esters (Tween ester), polyoxyethylene monoalkyl ethers, lanolin ethers, lanolin esters, acetic acid, hydrochloric acid, ammonium hydroxide, ammonium carbonate, potassium hydroxide, sodium hydroxide, prolamine, polyvinyl pyrrolidone, ethyl cellulose, carboxymethyl cellulose sodium, and the like may be used as the additives for liquid formulations according to the present invention.

A solution of white sugar, other sugars, a sweetening agent, and the like may be used as the syrup according to the present invention. A fragrance, a coloring agent, a preservative, a stabilizing agent, a suspending agent, an emulsifier, a thickening agent, and the like may be used, when necessary.

Purified water may be used in the emulsion according to the present invention and an emulsifier, a preservative, a stabilizing agent, a fragrance, and the like may be used, when necessary.

Suspending agents such as acacia, tragacanth, methyl cellulose, carboxymethyl cellulose, carboxymethyl cellulose sodium, microcrystalline cellulose, sodium alginate, hydroxypropyl methyl cellulose, HPMC 1828, HPMC 2906, HPMC 2910, and the like may be used in the suspension according to the present invention. A surfactant, a preservative, a stabilizing agent, a coloring agent, and a fragrance may be used, when necessary.

The injection according to the present invention may include solvents such as distilled water for injection, a 0.9% sodium chloride injection solution, Ringer's injection solution, a dextrose injection solution, a dextrose+sodium chloride injection solution, PEG, a lactated Ringer's injection solution, ethanol, propylene glycol, non-volatile oil-sesame oil, cottonseed oil, peanut oil, soybean oil, corn oil, ethyl oleate, isopropyl myristate, benzyl benzoate, and the like; solubilizing aids such as sodium benzoate, sodium salicylate, sodium acetate, urea, urethane, monoethylacetamide, butazolidine, propylene glycol, Tweens, nicotinamide, hexamine, dimethylacetamide, and the like; buffering agents such as a weak acid and salts thereof (acetic acid and sodium acetate), a weak base and salts thereof (ammonia and ammonium acetate), an organic compound, a protein, albumin, peptone, gum, and the like; isotonic agents such as sodium chloride and the like; stabilizing agents such as sodium bisulfite (NaHSO₃) carbon dioxide gas, sodium metabisulfite (Na₂S₂O₅), sodium sulfite (Na₂SO₃), nitrogen gas (N₂), ethylene diamine tetraacetic acid, and the like; antioxidants such as sodium bisulfide (0.1%), sodium formaldehyde sulfoxylate, thiourea, ethylene diamine disodium tetraacetate, acetone sodium bisulfite, and the like; analgesics such as benzyl alcohol, chlorobutanol, procaine hydrochloride, glucose, calcium gluconate, and the like; suspending agents such as CMC sodium, sodium alginate, Tween 80, aluminum monostearate, and the like.

Bases such as cacao butter, lanolin, Witepsol, polyethylene glycol, glycerogelatin, methyl cellulose, carboxymethyl cellulose, a mixture of stearic acid and oleic acid, Subanal, cottonseed oil, peanut oil, palm oil, cacao butter+cholesterol, lecithin, lanette wax, glycerol monostearate, Tween or Span, Imhausen, monolene (propylene glycol monostearate), glycerin, Adeps solidus, Buytyrum Tego-G, Cebes Pharma 16, Hexaride Base 95, Cotomar, Hydrokote SP, S-70-XXA, S-70-XX75 (S-70-XX95), Hydrokote 25, Hydrokote 711, Idropostal, Massa estrarium A, AS, B, C, D, E, I, T, Massa-MF, Masuppol, Masuppol-15, Neosuppostal-N, Paramound-B, Supposiro (OSI, OSIX, A, B, C, D, H, L), a suppository base type IV (AB, B, A, BC, BBG, E, BGF, C, D, 299), Suppostal (N, Es), Wecoby (W, R, S, M, Fs), a Tegester triglyceride base (TG-95, MA, 57), and the like may be used in a suppository according to the present invention.

A solid preparation for oral administration may include a tablet, a pill, a powder, granules, a capsule, and the like. Such a solid preparation is prepared by mixing at least one excipient, for example, starch, calcium carbonate, sucrose or lactose, gelatin, and the like with an extract. Also, lubricants such as magnesium stearate, talc, and the like are also used in addition to the simple excipients.

A liquid preparation for oral administration may include a suspension, an oral liquid, an emulsion, syrup, and the like. In addition to the commonly used simple diluents such as water, liquid paraffin, and the like, the liquid preparation may include various excipients, for example, a wetting agent, a sweetening agent, a fragrance, a preservative, and the like.

A preparation for parenteral administration includes a sterile aqueous solution, a non-aqueous solution, a suspension, an emulsion, a freeze-dried preparation, a suppository, and the like. Propylene glycol, polyethylene glycol, a vegetable oil (such as olive oil), an injectable ester (such as ethyl oleate), and the like may be used as a non-aqueous solvent and a suspending agent.

The pharmaceutical composition according to the present invention is administered at a pharmaceutically effective amount. In the present invention, the term “pharmaceutically effective amount” refers to an amount sufficient to treat a disease at a reasonable benefit/risk ratio applicable to medical treatment, and a level of an effective dose may be determined according to the type and severity of a patient's disease, the activity of a drug, the sensitivity to the drug, an administration duration, a route of administration and a secretion rate, a treatment period, factors including a concurrently used drug, and other factors well known in the medical field.

The pharmaceutical composition according to the present invention may be administered as an individual therapeutic agent or administered in combination with other therapeutic agents. In this case, the pharmaceutical composition may be administered sequentially or simultaneously with conventional therapeutic agents, and may be administered in single or multiple doses. It is important to administer an amount of the pharmaceutical composition that may realize the maximum effect at the minimum amount without any side effects in consideration of all the factors described above. In this case, the amount of the pharmaceutical composition may be easily determined by those skilled in the art to which the present invention belongs.

The pharmaceutical composition of the present invention may be administered to a subject via various routes of administration. All modes of administration may be expected, and the pharmaceutical composition may be, for example, administered by oral intake, subcutaneous injection, intraperitoneal administration, intravenous injection, intramuscular injection, paravertebral (intradural) injection, sublingual administration, buccal administration, transrectal administration, cervical insertion, intraocular administration, intraauricular administration, intranasal administration, inhalation, spraying through the mouth or nose, transdermal administration, dermal administration, and the like.

In the pharmaceutical composition of the present invention, the mode of administration may be determined according to the type of drug as an active ingredient in combination with various related factors such as a disease to be treated, a route of administration, the age, gender, and weight of a patient, the severity of a disease, and the like.

In the present invention, the term “subject” refers to a target in need of disease treatment. More specifically, the subject may include mammals such as a human or a non-human primate, a mouse, a rat, a dog, a cat, a horse, cattle, and the like, but the present invention is not limited thereto.

In the present invention, the term “administration” refers to a process of providing a predetermined amount of the composition of the present invention to a subject using any suitable method.

In the present invention, the term “prevention” refers to all types of actions for suppressing or delaying the onset of a target disease, and the term “treatment” refers to all types of actions for alleviating or benefiting a target disease and its symptoms associated with the metabolic abnormalities by administering the pharmaceutical composition according to the present invention. Also, the term “alleviation” refers to all types of actions for reducing at least the degree of parameters, for example, symptoms associated with the target disease by administering the composition of the present invention.

Also, the present invention may provide a food composition including the compound.

When the compound of the present invention is used as a food additive, the compound may be added as it is, or may be used in conjunction with other foods or food ingredients. In this case, the compound may be properly used according to conventional methods. A mixing amount of the active ingredient may be properly determined according to the purpose of use (prophylactic, healthcare, or therapeutic treatment). In general, when a food or a drink is prepared, the compound of the present invention may be added at an amount of 15% by weight or less, or 10% by weight or less, based on the weight of the raw materials. However, in the case of long-term intake for the purpose of health and hygiene or the purpose of health control, the amount of the active ingredient may be less than the described-above range. Also, the active ingredient may be used at an amount greater than the range because the active ingredient has no problem in terms of safety.

Types of the food are not particularly limited. Examples of foods to which the material may be added include meat, sausage, bread, chocolate, candies, snacks, confectioneries, pizza, ramen, other noodles, gum, dairy products (including ice cream), various soups, beverages, tea, drinks, alcoholic drinks, vitamin complexes, and the like. In this case, the foods include all types of health functional foods in the ordinary sense.

A health drink composition according to the present invention may contain various additional components such as flavoring agents, natural carbohydrates, or the like. The natural carbohydrates include monosaccharides such as glucose and fructose; disaccharides such as maltose and sucrose; polysaccharides such as dextrin and cyclodextrin; sugar alcohols such as xylitol, sorbitol, and erythritol; and the like. Natural sweetening agents such as thaumatin, a Stevia extract, and the like; or synthetic sweetening agents such as saccharine, aspartame, and the like may be used as the sweetening agent. The proportion of the natural carbohydrate is generally in a range of approximately 0.01 to 0.20 g, or approximately 0.04 to 0.10 g per 100 mL of the composition of the present invention.

In addition, the composition of the present invention may contain various nutrients, vitamins, electrolytes, a flavoring agent, a coloring agent, pectic acid or a salt thereof, alginic acid or a salt thereof, organic acids, a protective colloidal thickening agent, a pH controller, a stabilizing agent, a preservative, glycerin, an alcohol, a carbonating agent used in carbonated drinks, or the like. Also, the composition of the present invention may contain flesh for preparation of natural fruit juices, fruit juice drinks, and vegetable drinks. These components may be used alone or in combination. The proportions of such additives are not very important, but are generally selected from a range of 0.01 to 0.20 parts by weight based on 100 parts by weight of the composition of the present invention.

Also, the composition of the present invention may be provided in the form of a cosmetic composition.

The cosmetic composition may be prepared into forms such as a toner, a face lotion, a nourishing essence, a massage cream, a beauty bath additive, a body lotion, a body milk, a bath oil, a baby oil, a baby powder, a shower gel, a shower cream, a sunscreen lotion, a sunscreen cream, a suntan cream, a skin lotion, a skin cream, sunblock cosmetics, a cleansing milk, depilatories, a face and body lotion, a face and body cream, a skin-whitening cream, a hand lotion, a hair lotion, a cosmetic cream, jasmine oil, a bath soap, a liquid soap, a beauty soap, a shampoo, a hand sanitizer (a hand cleaner), a non-medicated soap, a cream soap, a facial wash, a body cleanser, a scalp cleanser, a hair rinse, a beauty soap, a tooth-whitening gel, a toothpaste, and the like. The composition of the present invention may further include a solvent, or a suitable carrier, excipient or diluent typically used for preparing cosmetic compositions.

Types of solvents that may be further added into the cosmetic composition of the present invention are not particularly limited. For example, water, saline, DMSO, or a combination thereof may be used. Also, the carrier, excipient, or diluent may include purified water, an oil, a wax, a fatty acid, a fatty acid alcohol, a fatty acid ester, a surfactant, a humectant, a thickening agent, an antioxidant, a viscosity stabilizing agent, a chelating agent, a buffer, a lower alcohol, and the like, but the present invention is not limited thereto. Also, the composition may include a whitening agent, a moisturizing agent, vitamins, a sunscreen, a fragrance, a dye, an antibiotic, an antibacterial agent, an antifungal agent, and the like.

A vegetable oil, castor oil, cottonseed oil, olive oil, palm-kernel oil, jojoba oil, avocado oil, and the like may be used as the oil of the present invention, and beeswax, spermaceti, carnauba, candelilla, montan, ceresin, liquid paraffin, lanolin, and the like may be used as the wax.

Stearic acid, linoleic acid, linolenic acid, oleic acid, and the like may be used as the fatty acid of the present invention, cetyl alcohol, octyl dodecanol, oleyl alcohol, panthenol, lanolin alcohol, stearyl alcohol, hexadecanol, and the like may be used as the fatty acid alcohol, and isopropyl myristate, isopropyl palmitate, butyl stearate, and the like may be used as the fatty acid ester. A cationic surfactant, an anionic surfactant, and a non-ionic surfactant known in the art may be used as the surfactant. In this case, natural product-derived surfactants are preferred if possible. In addition, the composition may include a humectant, a thickening agent, an antioxidant, and the like widely known in the field of cosmetics. In this case, types and amounts of the described-above additives are known in the art.

Throughout the specification of the present invention, when any certain part is said to “include” any component, this means that it may further include other components, rather than excluding other components unless otherwise stated. In addition, the terms “approximately,” “substantially,” the like used throughout the specification of the present invention are used at, or in close proximity to, numerical values when manufacturing and material tolerances inherent in the indicated meanings are intended to aid in understanding the present invention. Precise or absolute figures are used to assist in the prevention of unfair use by unscrupulous infringers. The terms “step of” used throughout the specification of the present invention does not refer to “step for”.

Throughout the specification of the present invention, the term “combination thereof” included in a Markush type expression refers to a mixture or combination of one or more selected from the group consisting of components disclosed in the Markush type expression, and thus means that the mixture or combination include one or more selected from the group consisting of the components.

Throughout the specification of the present invention, “A and/or B” refers to “A or B, or A and B”.

### Mode for Invention

Hereinafter, the present invention will be described in detail with reference to exemplary embodiments thereof. However, it will be apparent to those skilled in the art that the following embodiments are given by way of illustration only to more easily understand the present invention, and are not intended to limit the present invention.

### EXAMPLES

### [Preparation Examples 1 to 16] Synthesis of Novel Compound

Preparation Examples 1 to 16 listed in Table 1 below were synthesized according to processes described in the following Preparation Processes 1 to 8.

TABLE 1

| Preparation | |
|---|---|
| Example | Formula |
| 1 | 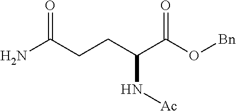 [SMILES: CC(=O)N[C@@H](CCC(N)=O)C(=O)OCc1ccccc1] |
| 2 | 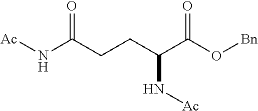 [SMILES: CC(=O)NC(=O)CC[C@H](NC(C)=O)C(=O)OCc1ccccc1] |
| 3 | 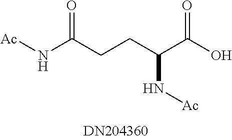 [SMILES: CC(=O)NC(=O)CC[C@H](NC(C)=O)C(=O)O] |
| 4 | 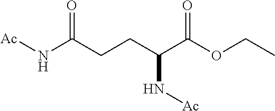 [SMILES: CCOC(=O)[C@H](CCC(=O)NC(C)=O)NC(C)=O] |
| 5 | 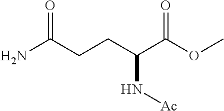 |
| 6 | 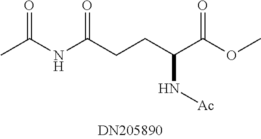 [SMILES: COC(=O)[C@H](CCC(=O)NC(C)=O)NC(C)=O] |
| 7 | 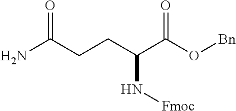 |
| 8 | 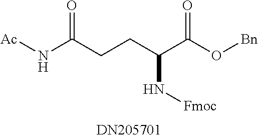 |
| 9 | 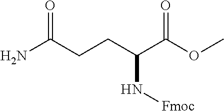 |
| 10 | 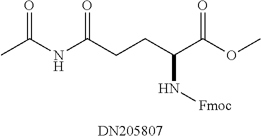 |
| 11 | 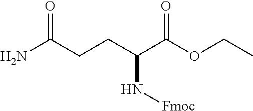 |
| 12 | 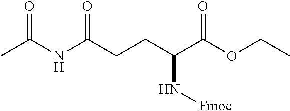 |
| 13 | 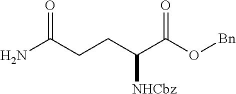 [SMILES: NC(=O)CC[C@H](NC(=O)OCc1ccccc1)C(=O)OCc1ccccc1] |
| 14 | 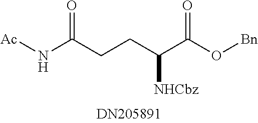 [SMILES: CC(=O)NC(=O)CC[C@H](NC(=O)OCc1ccccc1)C(=O)OCc1ccccc1] |
| 15 | 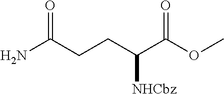 [SMILES: COC(=O)[C@H](CCC(N)=O)NC(=O)OCc1ccccc1] |
| 16 | 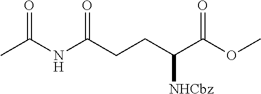 [SMILES: COC(=O)[C@H](CCC(=O)NC(C)=O)NC(=O)OCc1ccccc1] |

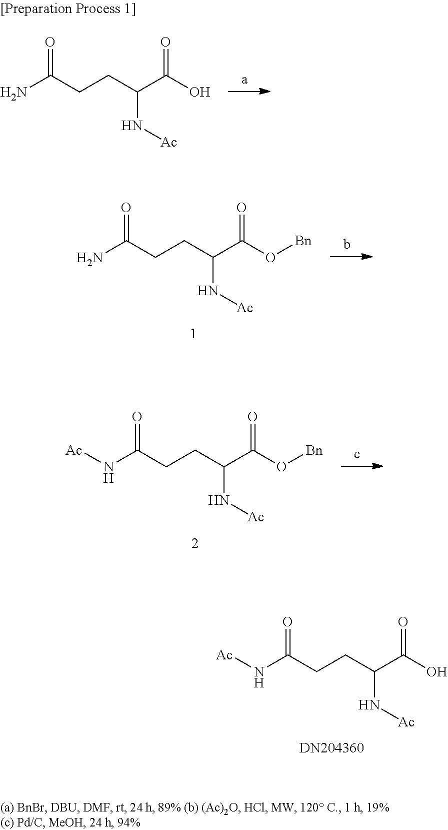 [SMILES: CC(=O)NC(CCC(N)=O)C(=O)O ; CC(=O)NC(CCC(N)=O)C(=O)OCc1ccccc1 ; CC(=O)NC(=O)CCC(NC(C)=O)C(=O)OCc1ccccc1 ; CC(=O)NC(=O)CCC(NC(C)=O)C(=O)O]

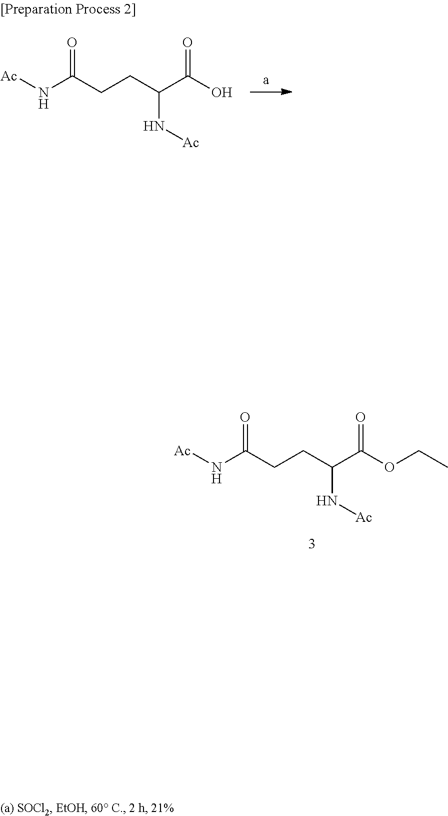 [SMILES: CC(=O)NC(=O)CCC(NC(C)=O)C(=O)O ; CCOC(=O)C(CCC(=O)NC(C)=O)NC(C)=O]

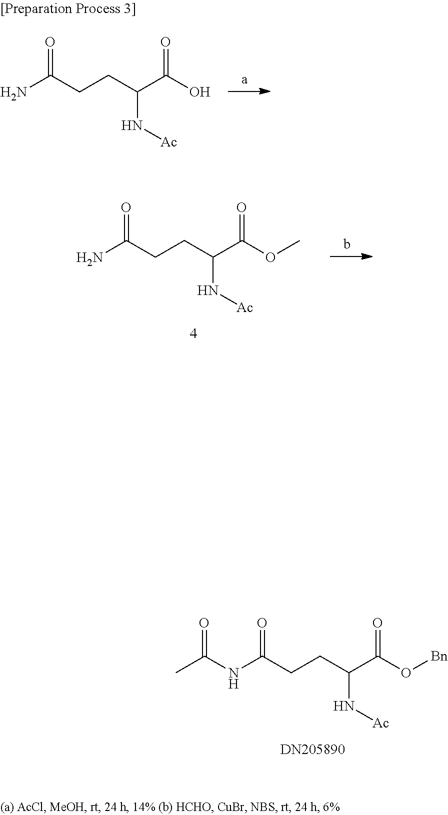 [SMILES: CC(=O)NC(CCC(N)=O)C(=O)O ; COC(=O)C(CCC(N)=O)NC(C)=O ; CC(=O)NC(=O)CCC(NC(C)=O)C(=O)OCc1ccccc1]

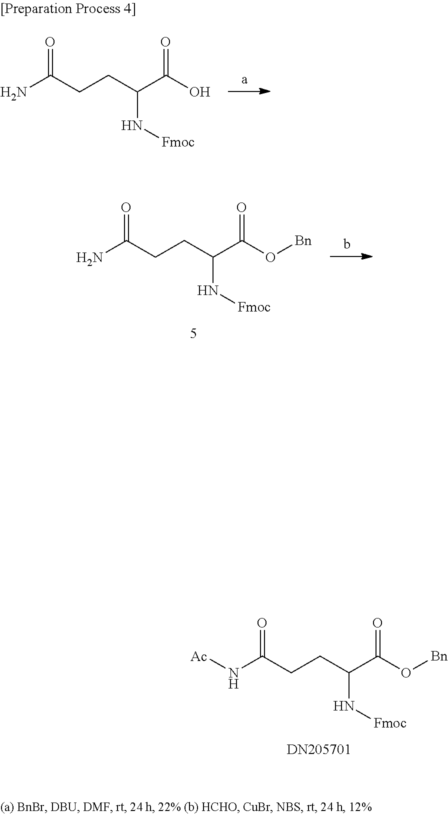

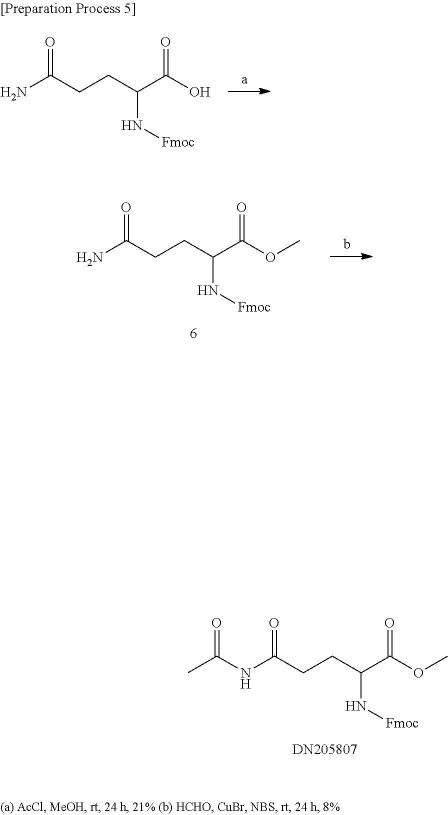

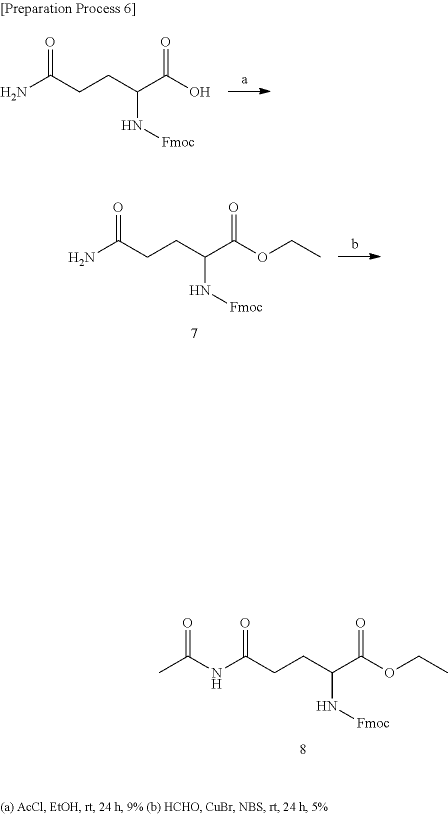

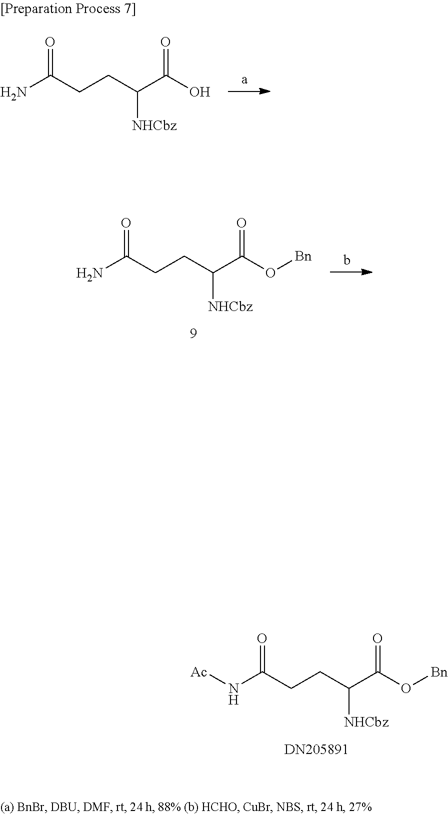 [SMILES: NC(=O)CCC(NC(=O)OCc1ccccc1)C(=O)O ; NC(=O)CCC(NC(=O)OCc1ccccc1)C(=O)OCc1ccccc1 ; CC(=O)NC(=O)CCC(NC(=O)OCc1ccccc1)C(=O)OCc1ccccc1]

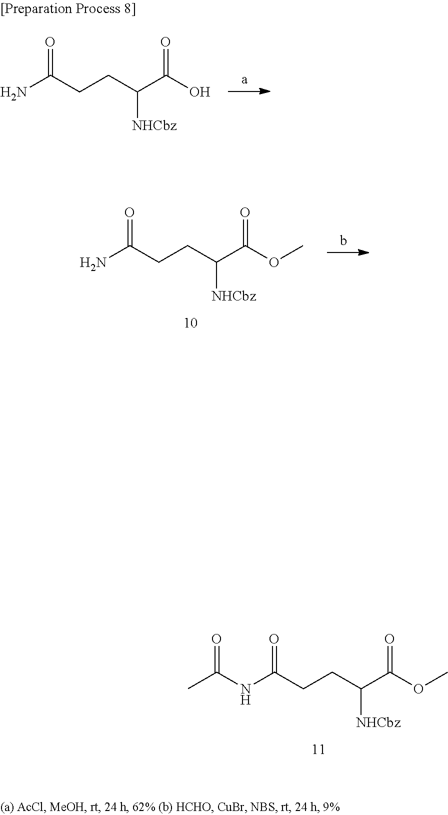 [SMILES: NC(=O)CCC(NC(=O)OCc1ccccc1)C(=O)O ; COC(=O)C(CCC(N)=O)NC(=O)OCc1ccccc1 ; COC(=O)C(CCC(=O)NC(C)=O)NC(=O)OCc1ccccc1]

### [Preparation Example 1] Benzyl 2-acetamido-5-amino-5-oxopentanoate

2-acetamido-5-amino-5-oxopentanoic acid (26.6 mmol) and benzyl bromide (1.5 eq) were added to 100 mL of dimethylformamide (DMF), and stirred at room temperature. Thereafter, 1,8-diazabicyclo[5,4,0]undec-7-ene (DBU (1.2 eq)) was added, and the resulting mixture was stirred at room temperature for 24 hours after the dropwise addition. When the reaction was completed, the reaction was terminated using water, dichloromethane was additionally added to extract an organic layer. The organic layer was washed with saline, moisture was removed with sodium sulfite, and the solvent was distilled under reduced pressure to obtain a residue. The residue was recrystallized using hexane and ethyl acetate to obtain Preparation Example 1 as the target compound (see Preparation Process 1).

¹H NMR (400 MHz, MeOD) δ 7.43-7.27 (m, 5H), 5.19 (s, 2H), 4.46 (dd, J=9.1, 5.1 Hz, 1H), 2.36-2.24 (m, 2H), 2.26-2.12 (m, 1H), 2.00 (s, 3H), 1.99-1.85 (m, 1H)

### [Preparation Example 2] Benzyl 2,5-diacetamido-5-oxopentanoate

Preparation Example 1 (23.0 mmol), acetaldehyde (3.0 eq), and hydrochloric acid (0.001 eq) were put into a microwave tube, and stirred at 50 W and 120° C. for 30 minutes. Thereafter, an organic layer was extracted using ethyl acetate and water, washed with saline, and then dried over sodium sulfite to remove moisture. Then, the remaining solvent was distilled under reduced pressure to obtain a residue. The finally obtained residue was separated and purified using MPLC to obtain Preparation Example 2 (see Preparation Process 1).

¹H NMR (400 MHz, DMSO-d₆) δ 10.63 (s, 1H), 8.30 (d, J=7.5 Hz, 1H), 7.47-7.23 (m, 5H), 5.17-5.06 (m, 2H), 4.27 (ddd, J=9.3, 7.5, 5.4 Hz, 1H), 2.70-2.38 (m, 2H), 2.12 (s, 3H), 2.07-1.91 (m, 1H), 1.85 (s, 3H), 1.83-1.73 (m, 1H)

### [Preparation Example 3] 2,5-diacetamido-5-oxopentanoic acid

Preparation Example 2 (4.06 mmol) was dissolved in 5 mL of methanol, and 0.2 g of Pd/C (palladium on activated carbon) was also added thereto. The mixture was stirred for 24 hours under a hydrogen atmosphere. After the reaction was terminated, the reaction solution was filtered through Celite, and then distilled under reduced pressure to obtain Preparation Example 3 (DN204360) (see Preparation Process 1).

¹H NMR (400 MHz, DMSO-d₆) δ 10.64 (s, 1H), 8.13 (d, J=7.8 Hz, 1H), 4.15 (td, J=8.9, 5.1 Hz, 1H), 2.56-2.46 (m, 2H), 2.13 (s, 3H), 2.06-1.89 (m, 1H), 1.84 (s, 3H), 1.80-1.64 (m, 1H)

### [Preparation Example 4] Ethyl 2,5-diacetamido-5-oxopentanoate

2,5-diacetamido-5-oxopentanoic acid (0.2 mmol) was sufficiently dissolved in ethanol, and then stirred at 0° C. for 10 minutes, and 1.2 equivalents of thionyl chloride (SOCl₂) was further added thereto. Thereafter, the reactants were stirred at room temperature for 20 minutes, and then stirred at 60° C. for 2 hours. When the reaction was terminated, water was added, and dichloromethane was further added to the mixture to extract an organic layer. The organic layer was washed with saline, and dried with sodium sulfite to remove moisture, and the solvent was distilled under reduced pressure to obtain a residue. Then, the residue was separated and purified by Prep HPLC to obtain Preparation Example 4 (see Preparation Process 2).

¹H NMR (400 MHz, DMSO-d₆) δ 8.30 (d, J=7.4 Hz, 1H), 7.41 (s, 1H), 6.71 (s, 1H), 4.26-4.11 (m, 1H), 4.14-3.94 (m, 2H), 2.34-2.27 (m, 2H), 1.98-1.88 (m, 1H), 1.85 (s, 3H), 1.82-1.70 (m, 4H), 1.22-1.12 (m, 3H)

### [Preparation Example 5] Methyl 2-acetamido-5-amino-5-oxopentanoate

2-acetamido-5-amino-5-oxopentanoic acid (5.31 mmol) was dissolved in 25 mL of methanol, and 1.05 equivalents of acetyl chloride was added dropwise, and then stirred at room temperature for 24 hours. When the reaction was terminated, a sodium hydrogen carbonate (NaHCO₃) solution was added, and the solvent was distilled under reduced pressure to obtain a residue. Then, the residue was separated and purified by MPLC to obtain Preparation Example 5 (see Preparation Process 3).

¹H NMR (400 MHz, CDCl₃) δ 6.49 (s, 1H), 6.15 (s, 1H), 5.36 (s, 1H), 4.60 (td, J=8.7, 4.4 Hz, 1H), 2.49-2.26 (m, 2H), 2.26-2.11 (m, 1H), 2.13-1.83 (m, 4H)

### [Preparation Example 6] Methyl 2,5-diacetamido-5-amino-5-oxopentanoate

Acetaldehyde (0.3 mmol), Preparation Example 5 (1.2 eq), and CuBr (0.05 eq) were added to 6 mL of a solution of acetonitrile and methyl chloride mixed at a ratio of 1:5, and stirred at room temperature for 15 minutes. Thereafter, 1.5 equivalents of N-bromosuccinimide was added, and stirred at room temperature for 24 hours. When the reaction was terminated, an organic layer was extracted using ethyl acetate and water, washed with saline, and then dried over sodium sulfite to remove moisture. Then, the solvent was distilled under reduced pressure to obtain a residue. Then, the residue was separated and purified by MPLC to obtain Preparation Example 6 (DN205890) (see Preparation Process 3).

¹H NMR (400 MHz, MeOD) δ 4.58-4.32 (m, 1H), 3.72 (s, 3H), 2.75-2.42 (m, 2H), 2.29-2.07 (m, 4H), 2.07-1.80 (m, 4H)

### [Preparation Example 7] Benzyl 2-((((9H-fluoren-9-yl)methoxy)carbonyl)amino)-5-amino-5-oxopentanoate

The synthesis was performed in the same manner as in Preparation Example 1, using 2-((((9H-fluoren-9-yl)methoxy)carbonyl)amino)-5-amino-5-oxopentanoic acid as the starting material, to obtain Preparation Example 7 (see Preparation Process 4).

¹H NMR (400 MHz, CDCl₃) δ 7.77 (d, J=7.5 Hz, 2H), 7.57 (t, J=16.0 Hz, 2H), 7.43-7.14 (m, 9H), 5.77 (s, 1H), 5.64 (d, J=7.6 Hz, 1H), 5.31 (s, 1H), 5.19 (q, J=12.2 Hz, 2H), 4.41 (d, J=7.0 Hz, 3H), 4.21 (t, J=6.8 Hz, 1H), 2.24 (t, J=7.2 Hz, 3H), 1.99 (d, J=8.0 Hz, 1H)

### [Preparation Example 8] Benzyl 2-((((9H-fluoren-9-yl)methoxy)carbonyl)amino)-5-acetamido-5-oxopentanoate

The synthesis was performed in the same manner as in Preparation Example 6, using Preparation Example 7, to obtain Preparation Example 8 (DN205701) (see Preparation Process 4).

¹H NMR (400 MHz, CDCl₃) δ 7.99 (s, 1H), 7.77 (d, J=7.5 Hz, 2H), 7.57 (t, J=16.4 Hz, 2H), 7.49-7.19 (m, 9H), 5.50 (d, J=8.1 Hz, 1H), 5.19 (q, J=12.5 Hz, 2H), 4.42 (d, J=6.8 Hz, 3H), 4.21 (d, J=6.0 Hz, 1H), 2.54-2.48 (m, 1H), 2.35-2.27 (m, 4H), 2.05-1.91 (m, 1H)

### [Preparation Example 9] Methyl 2-((((9H-fluoren-9-yl)methoxy)carbonyl)amino)-5-amino-5-oxopentanoate

The synthesis was performed in the same manner as in Preparation Example 5, using 2-((((9H-fluoren-9-yl)methoxy)carbonyl)amino)-5-amino-5-oxopentanoic acid as the starting material, to obtain Preparation Example 9 (see Preparation Process 5).

¹H NMR (400 MHz, MeOD) δ 7.82-7.75 (m, 2H), 7.72-7.54 (m, 2H), 7.42-7.24 (m, 4H), 4.45-4.31 (m, 2H), 4.29-4.21 (m, 2H), 3.81-3.58 (m, 4H), 2.36-2.29 (m, 2H), 2.22-2.12 (m, 1H), 1.99-1.84 (m, 1H)

### [Preparation Example 10] Methyl 2-((((9H-fluoren-9-yl)methoxy)carbonyl)amino)-5-acetamido-5-oxopentanoate

The synthesis was performed in the same manner as in Preparation Example 6, using Preparation Example 9, to obtain Preparation Example 10 (DN205807) (see Preparation Process 5).

¹H NMR (400 MHz, CDCl₃) δ 8.73 (s, 1H), 7.76 (d, J=7.5 Hz, 2H), 7.58 (t, J=9.6 Hz, 2H), 7.40 (t, J=7.4 Hz, 2H), 7.30 (dd, J=17.3, 9.9 Hz, 2H), 5.62 (d, J=8.1 Hz, 1H), 4.42 (t, J=10.3 Hz, 3H), 4.21 (t, J=6.8 Hz, 1H), 3.76 (s, 3H), 2.61 (d, J=5.5 Hz, 2H), 2.37-2.16 (m, 4H), 1.98 (dd, J=14.3, 7.4 Hz, 1H)

### [Preparation Example 11] Ethyl 2-((((9H-fluoren-9-yl)methoxy)carbonyl)amino)-5-amino-5-oxopentanoate

2-((((9H-fluoren-9-yl)methoxy)carbonyl)amino)-5-amino-5-oxopentanoic acid (2.71 mmol) was dissolved in 25 mL of ethanol, and 1.05 equivalents of acetyl chloride was added dropwise, and then stirred at room temperature for 24 hours. When the reaction was terminated, a sodium hydrogen carbonate (NaHCO₃) solution was added thereto, and the solvent was distilled under reduced pressure to obtain a residue. The residue was separated and purified by MPLC to obtain Preparation Example 11 (see Preparation Process 6).

¹H NMR (400 MHz, MeOD) δ 7.70 (d, J=7.5 Hz, 2H), 7.63-7.45 (m, 2H), 7.29 (t, J=7.4 Hz, 2H), 7.22 (t, J=7.4 Hz, 2H), 4.37-4.18 (m, 2H), 4.18-3.95 (m, 4H), 2.30 (t, J=7.4 Hz, 2H), 2.05 (dt, J=13.3, 7.6 Hz, 1H), 1.81 (dd, J=14.0, 8.7 Hz, 1H), 1.28-1.01 (m, 3H)

### [Preparation Example 12] Ethyl 2-((((9H-fluoren-9-yl)methoxy)carbonyl)amino)-5-acetamido-5-oxopentanoate

The synthesis was performed in the same manner as in Preparation Example 6, using Preparation Example 11, to obtain Preparation Example 12 (see Preparation Process 6).

¹H NMR (400 MHz, MeOD) δ 7.82 (d, J=7.5 Hz, 2H), 7.71 (dd, J=15.2, 8.7 Hz, 2H), 7.61 (s, 1H), 7.41 (t, J=7.4 Hz, 2H), 7.33 (t, J=7.4 Hz, 2H), 4.50-4.30 (m, 2H), 4.30-4.06 (m, 4H), 2.63 (t, J=7.2 Hz, 2H), 2.28-2.12 (m, 4H), 1.95 (dt, J=16.0, 7.2 Hz, 1H), 1.40-1.15 (m, 2H)

### [Preparation Example 13] Benzyl 5-amino-2-(((benzyloxy)carbonyl)amino)-5-oxopentanoate

The synthesis was performed in the same manner as in Preparation Example 1, using 5-amino-2-(((benzyloxy)carbonyl)amino)-5-oxypentanoic acid as the starting material, to obtain Preparation Example 13 (see Preparation Process 7).

¹H NMR (400 MHz, CDCl₃) δ 7.57-7.06 (m, 10H), 5.95-5.53 (m, 2H), 5.41 (s, 1H), 5.23-4.99 (m, 4H), 4.41 (d, J=7.2 Hz, 1H), 2.25 (t, J=16.1 Hz, 2H), 2.10-1.91 (m, 1H), 1.86-1.79 (m, 1H)

### [Preparation Example 14] Benzyl 5-acetamido-2-(((benzyloxy)carbonyl)amino)-5-oxopentanoate

The synthesis was performed in the same manner as in Preparation Example 6, using Preparation Example 13, to obtain Preparation Example 14 (DN205891) (see Preparation Process 7).

¹H NMR (400 MHz, CDCl₃) δ 8.39 (s, 1H), 7.68-6.90 (m, 10H), 5.56 (d, J=7.9 Hz, 1H), 5.27-4.96 (m, 4H), 4.46 (d, J=4.6 Hz, 1H), 2.73-2.40 (m, 2H), 2.35-2.11 (m, 4H), 2.07-1.83 (m, 1H)

### [Preparation Example 15] Methyl 5-amino-2-(((benzyloxy)carbonyl)amino)-5-oxopentanoate

The synthesis was performed in the same manner as in Preparation Example 5, using 5-amino-2-(((benzyloxy)carbonyl)amino)-5-oxypentanoic acid as the starting material, to obtain Preparation Example 15 (see Preparation Process 8).

¹H NMR (400 MHz, MeOD) δ 7.58-6.99 (m, 5H), 5.20-5.00 (m, 2H), 4.22 (dd, J=9.1, 5.0 Hz, 1H), 3.74 (s, 3H), 2.33 (t, J=7.5 Hz, 2H), 2.16 (dt, J=13.0, 7.6 Hz, 1H), 1.93 (dt, J=22.1, 7.8 Hz, 1H)

### [Preparation Example 16] Methyl 5-acetamido-2-(((benzyloxy)carbonyl)amino)-5-oxopentanoate

The synthesis was performed in the same manner as in Preparation Example 6, using Preparation Example 15, to obtain Preparation Example 16 (see Preparation Process 8).

¹H NMR (400 MHz, MeOD) δ 7.65-7.16 (m, 5H), 5.20-4.98 (m, 2H), 4.26 (dd, J=9.3, 5.1 Hz, 1H), 3.74 (s, 3H), 2.63 (t, J=7.2 Hz, 2H), 2.29-2.07 (m, 4H), 1.94 (dd, J=15.1, 8.1 Hz, 1H)

### [Preparative Example 1] Breeding of Laboratory Animals

Eight-week-old female C56BL/6 mice (Orient Bio Korea Inc., Korea) having no certain pathogen were accommodated in an aseptic laminar-flow cabinet, and bred by supplying a standard solid feed ad libitum. When the following experiment was performed, the mice were made to correspond to 7- to 8-weeks old, and all laboratory animals were managed according to the protocol approved by the Institutional Animal Care and Use Committee of the Chonbuk National University Medical School.

[Experimental Method 1] Establishment of Asthma and Chronic Obstructive Pulmonary Disease-Induced Animal Models

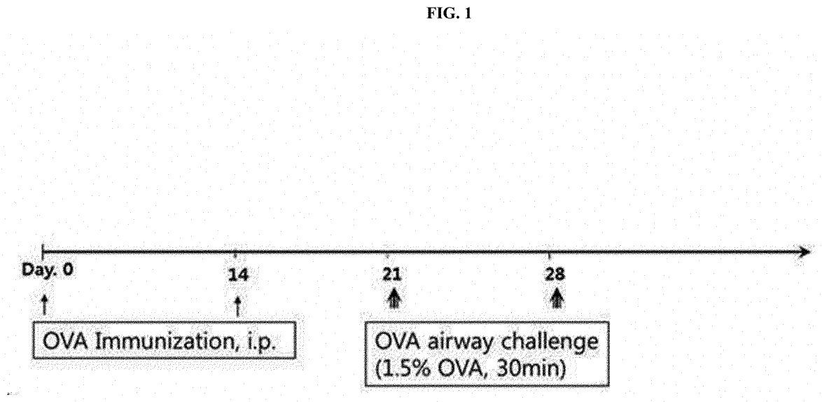

Establishment of asthma-induced animal model: As shown in FIG. 1, 20 μg of ovalbumin (OVA, grade V, Sigma Aldrich, USA) was mixed with 1.0 mg of an aluminum hydroxide adjuvant (Imject Alum; Pierce USA), and the resulting mixture was intraperitoneally injected on the experimental starting date (day 0) and day 14 (day 14) from the experimental starting date to perform a primary challenge. Also, on days 21 and 28 from the experimental starting date, the mice were put into a plastic box (Plexiglas (Rohm & Haas, Philadelphia, Pa.) exposure chamber; 24.5 cm×40.5 cm×15.0 cm), and 1.5% ovalbumin was evaporated using an ultrasonic nebulizer (NE-U12; output 0.8 mL/min; Omron, Japan), and injected into the box for 30 minutes so that the mice inhaled the evaporated ovalbumin (secondary challenge).

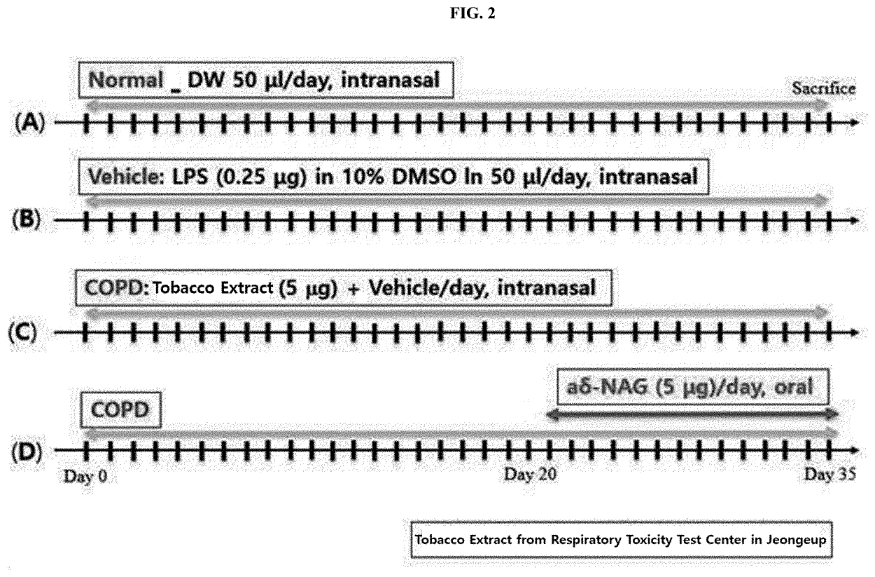

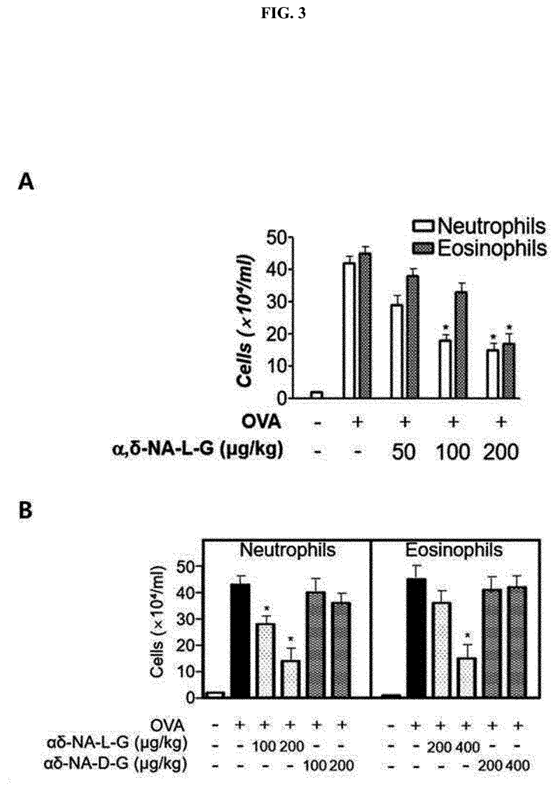

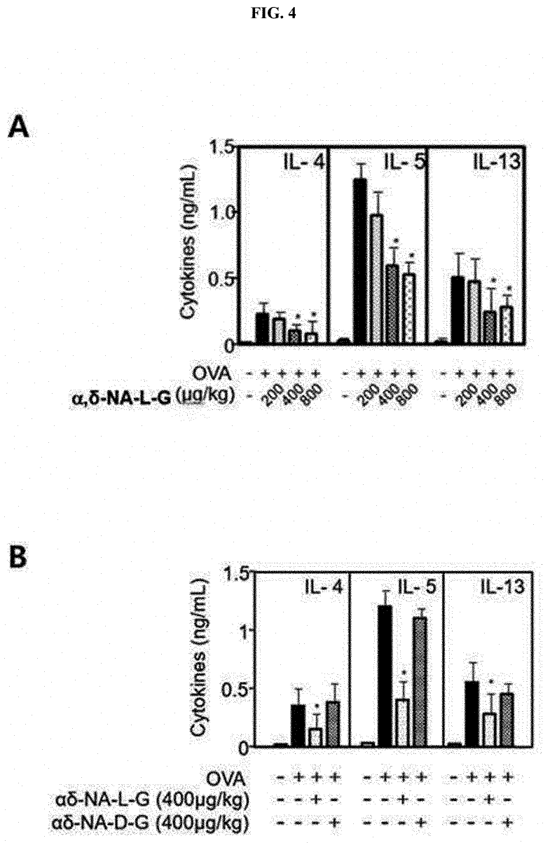

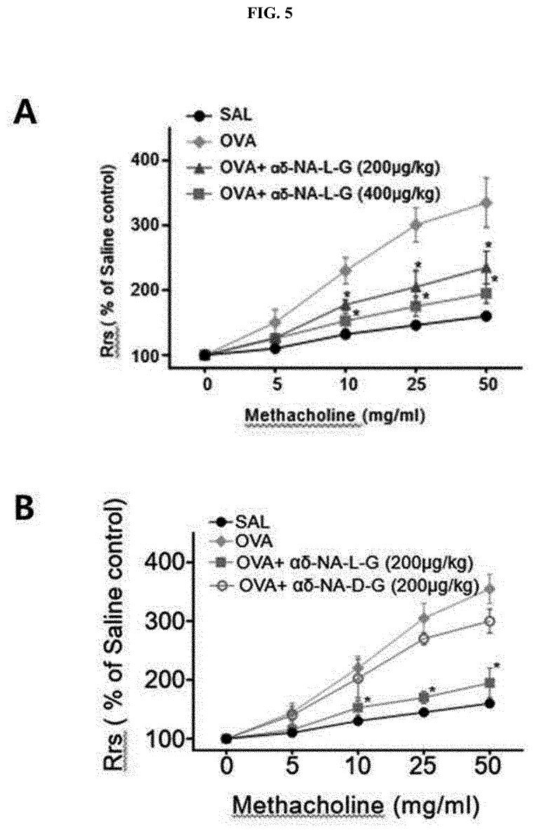

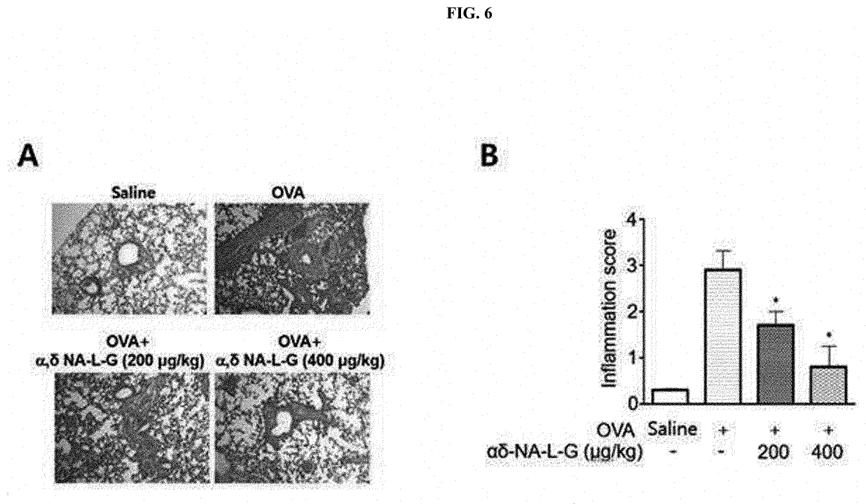

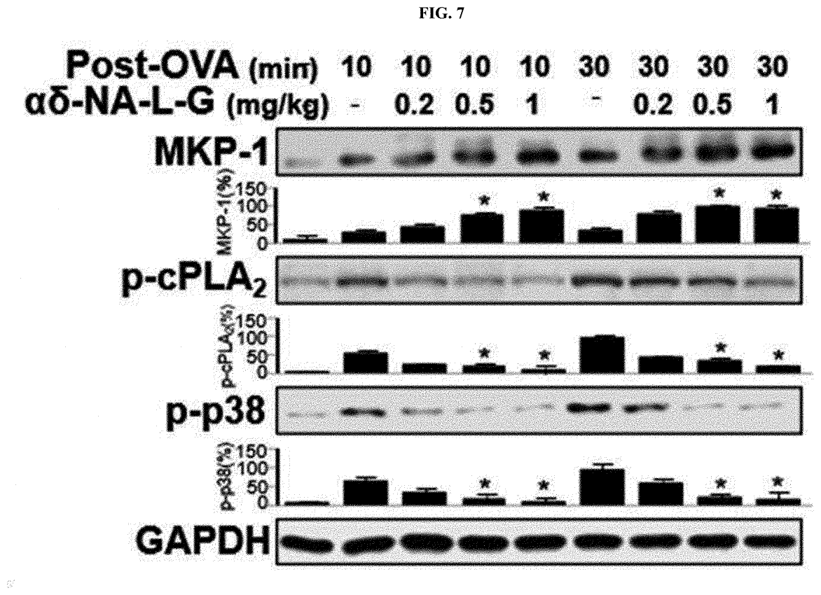

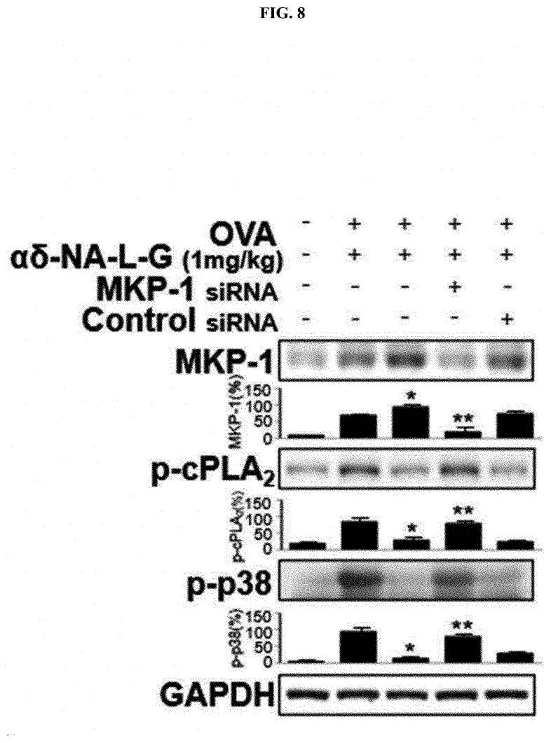

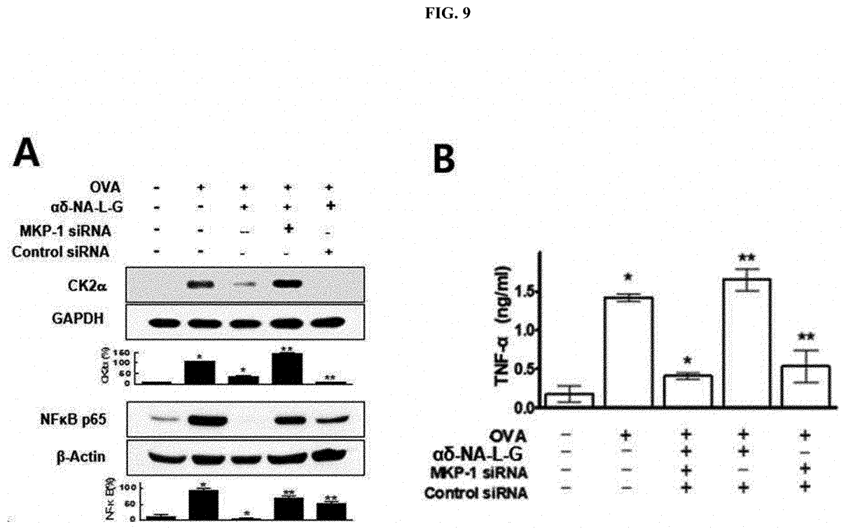

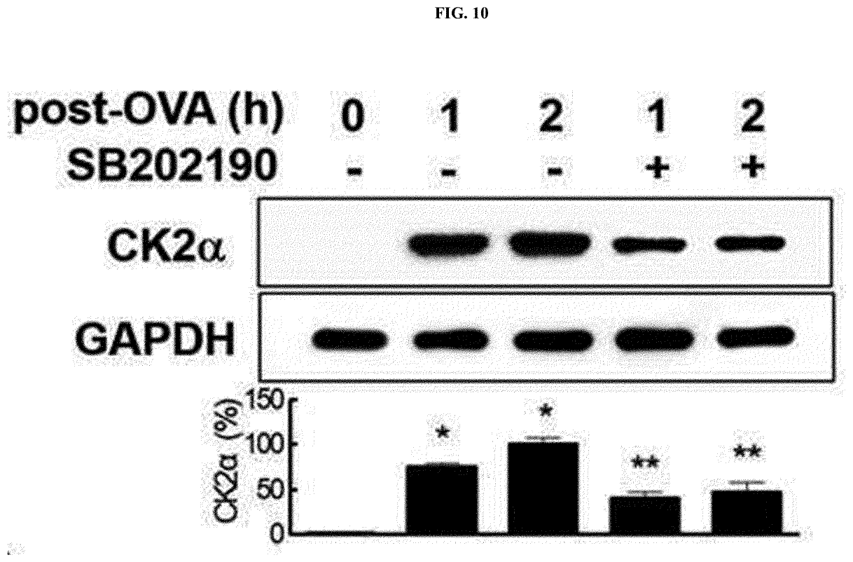

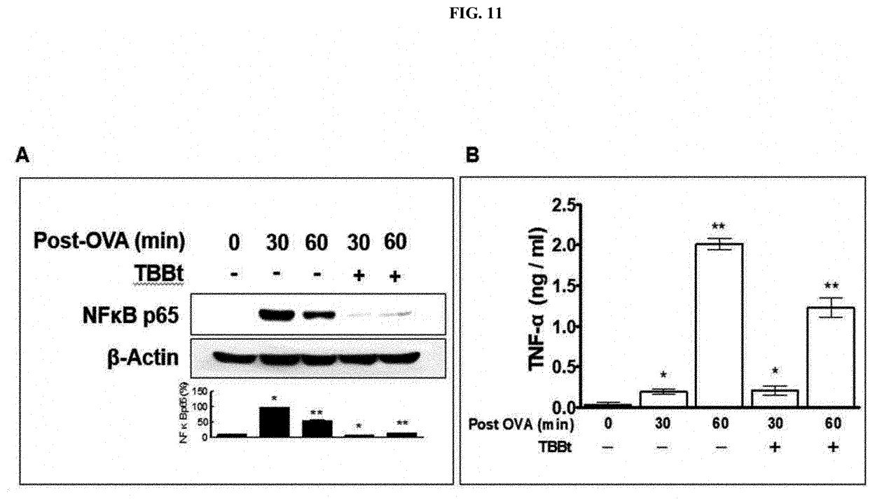

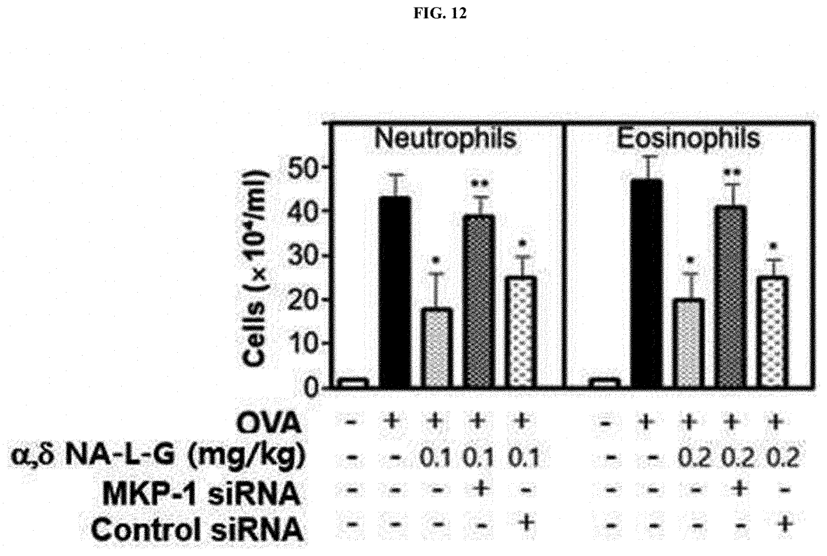

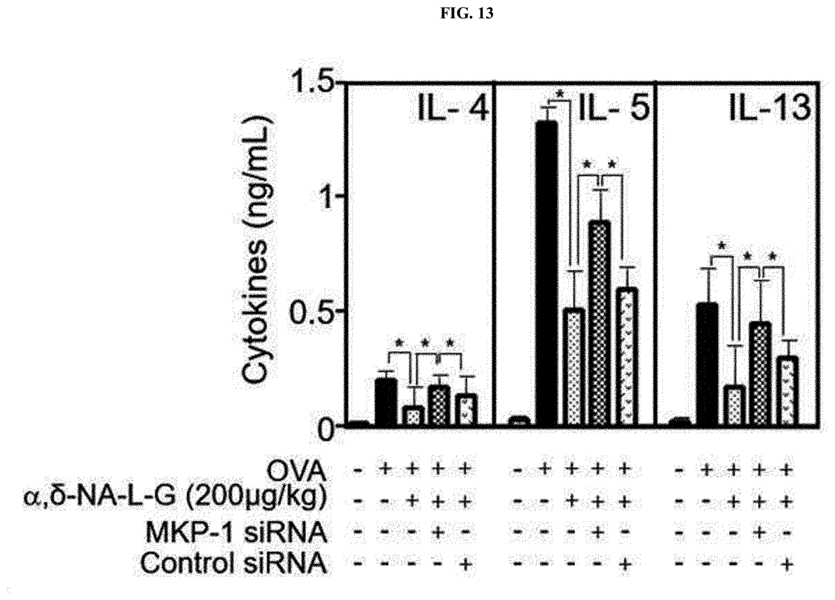

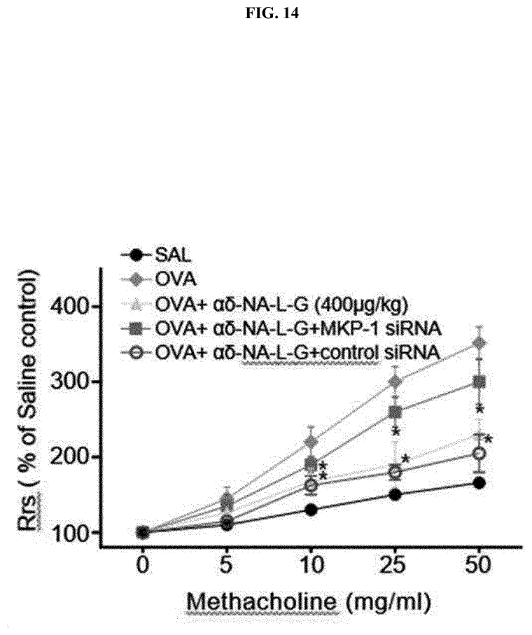

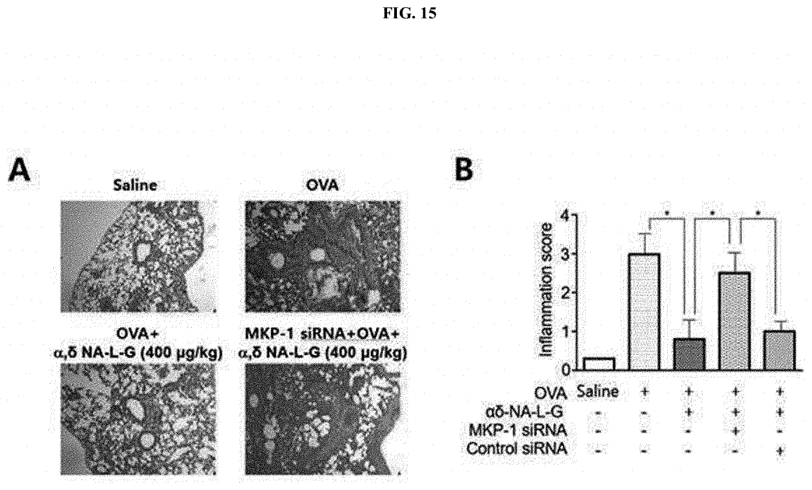

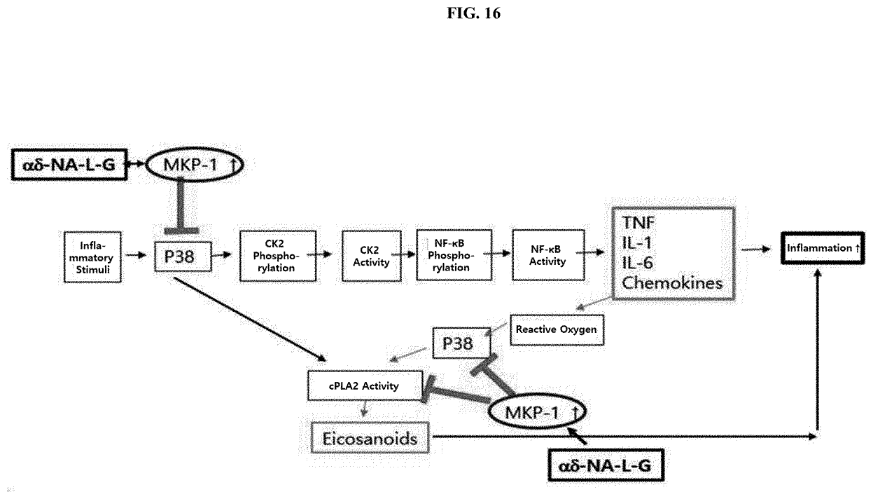

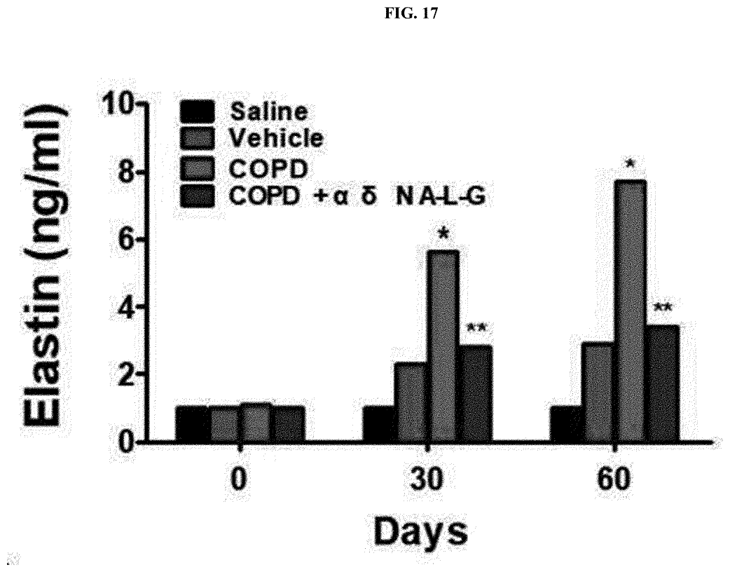

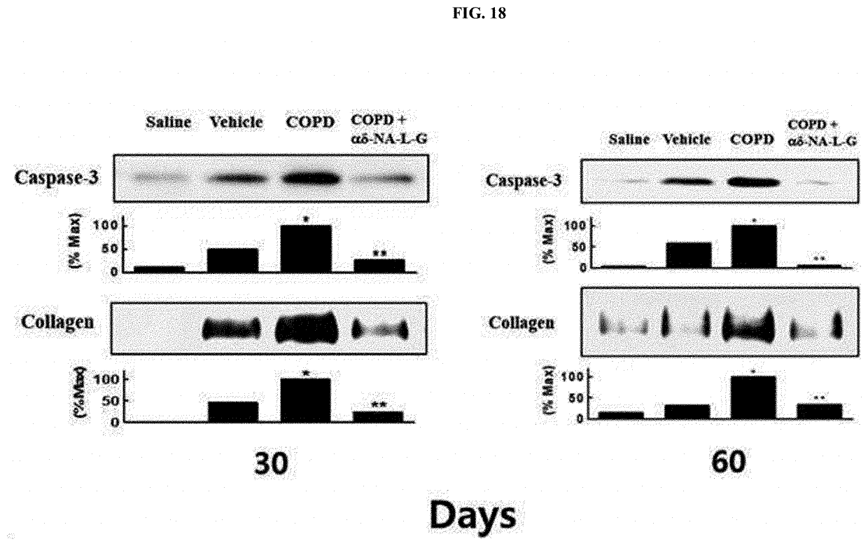

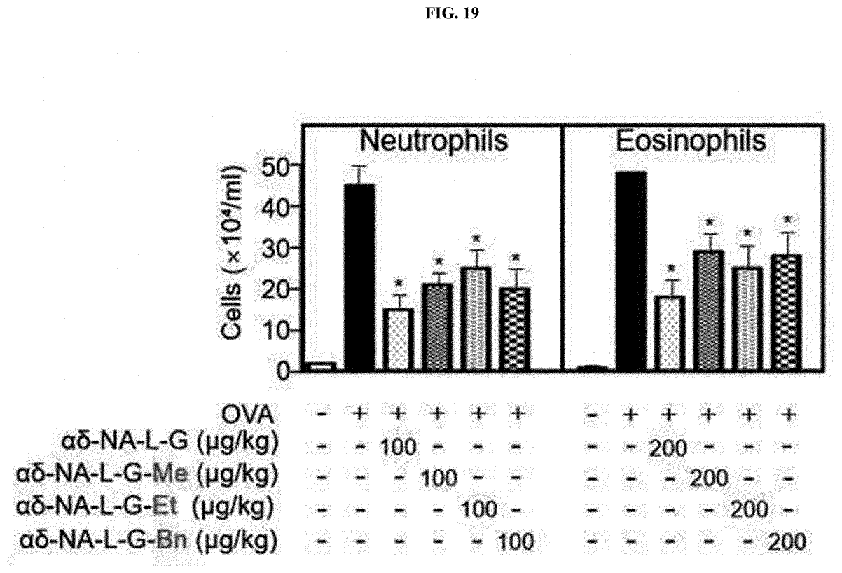

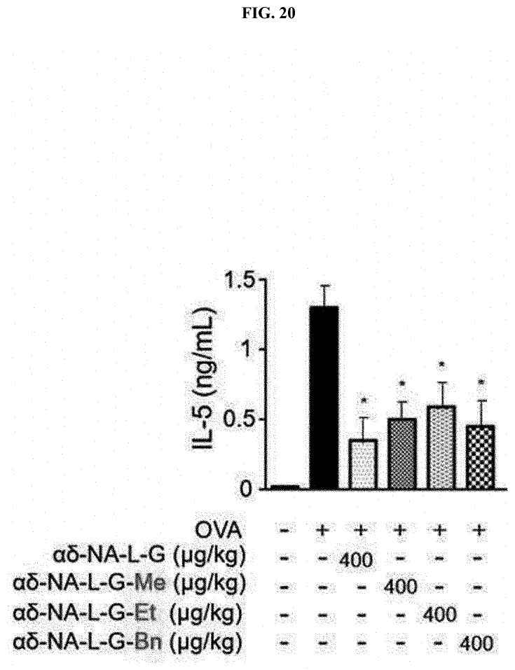

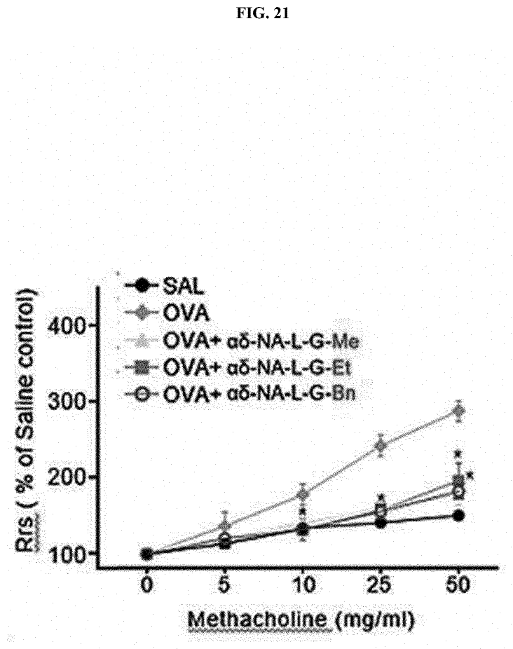

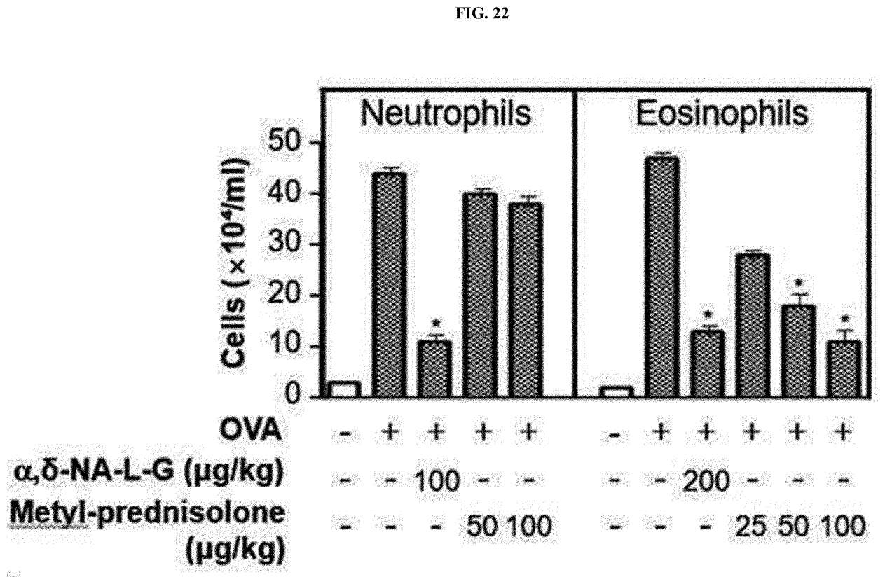

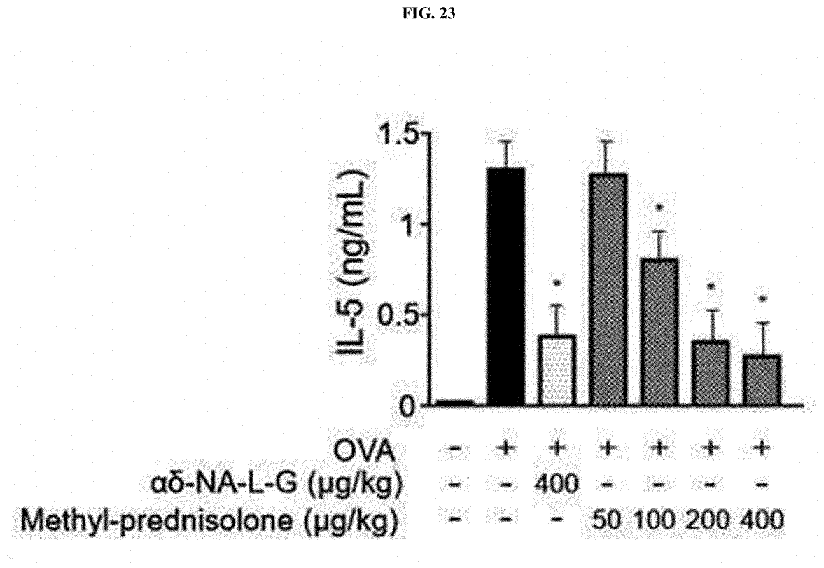

Establishment of chronic obstructive pulmonary disease-induced animal model: As shown in FIG. 2, a mixed solution obtained by mixing 5 μg of a tobacco extract (Respiratory Toxicity Test Center; Jeongeup-si, Jeollabuk-do, Korea) and 0.25 μg of LPS with 50 μL of a 10% DMSO solution was prepared. Then, the mixed solution was intranasally administered to the mice of Preparative Example 1 once a day for 30 days or 60 days from the experimental starting date. Here, a group to which only physiological saline was intranasally administered and a group to which a solution obtained by mixing 0.25 μg of LPS with 50 μL of a 10% DMSO solution was administered were used as the controls.

[Experimental Method 2] Collection of Bronchoalveolar Lavage Fluid

After the mice were anesthetized, a thin tube was inserted into the airway, and a piston process was repeated using a syringe filled with 0.2 mL of physiological saline to obtain a bronchoalveolar lavage fluid. The bronchoalveolar lavage fluid obtained through this piston process was stored at −70° C. when it was not immediately used in the experiments.

[Experimental Method 3] Observation of Inflammatory Cells Present in Bronchoalveolar Lavage Fluid

50 μL of the bronchoalveolar lavage fluid obtained in Experimental Method 2 was put on a slide used for the cytospin, and centrifuged for 30 minutes. Thereafter, Diff-Quik staining was performed, and inflammatory cells (neutrophils and eosinophils) were observed using a microscope.

[Experimental Method 4] Measurement of Cytokines Present in Bronchoalveolar Lavage Fluid

A level of cytokines in the bronchoalveolar lavage fluid obtained in Experimental Method 2 was measured using an ELISA kit. Here, ELISA kits specifically manufactured for TNF-α, IL-4, IL-5, and IL-13 were used.

[Experimental Method 5] Method of Confirming Bronchial Hypersensitivity Reaction

45 mg/kg of sodium pentobarbital was intraperitoneally injected into the mice prepared in Experimental Method 1 to anesthetize the mice, and the airways were cut and an 18 gauge needle was inserted into the airways. Thereafter, the needle was connected to a small-sized animal respirator connected to a computer, and the computer system was adjusted to a respiration rate of 150 breaths per minute. Then, methacholine in the form of aerosol was fed at an increasing concentration of 5.0 to 50 mg/mL so that the mice inhaled the methacholine. Then, an airway response was measured, and indicated as an Rrs value.

[Experimental Method 6] Lung Tissue Staining

A tissue was extracted from each of the mice manufactured in Experimental Method 1, and put into a 10% formalin solution, and reacted for 24 hours to prepare a paraffin block. Thereafter, the paraffin block was microtomed into slices with a size of 3 μm, and each of the slices was attached onto a slide, and heamatoxylin-eosin IHE staining was performed. Then, the slices were observed using a microscope. Here, the degree of inflammation was determined by indicating degrees of inflammation around the airway and blood vessels as grades 0 to 3.

0: There are no inflammatory cells;

1: Inflammatory cells are occasionally observed around the airway and blood vessels;

2: Most of the airway and blood vessels are surrounded by approximately 1 to 5 thin bands of inflammatory cells; and

3: Most of the airway and blood vessels are surrounded by thick bands of inflammatory cells (5 or more).

[Experimental Method 7] Method of Measuring Expression Level of Protein

A tissue was extracted from each of the mice manufactured in Experimental Method 1, and finely ground to a cell level. Thereafter, the cells were lysed in a cell lysis buffer containing a protease inhibitor to obtain a cell lysate. The cell lysate was centrifuged to obtain protein. Then, an equivalent amount of the proteins obtained from the cells were loaded on an SDS-PAGE gel and electrophoresed. The proteins present in the electrophoresed gel were transferred to a PVDF membrane. The PVDF membrane was washed, and added to a blocking buffer in which 5% skim milk was included in a TBS-T buffer, and incubated at room temperature. Then, the PVDF membrane was incubated at room temperature with an antibody diluted 1:1,000 in a TBS-T buffer containing 3% BSA. Then, the PVDF membrane was washed with a TBS-T buffer, and incubated at room temperature for an hour with a dilute solution containing HRP-bound IgG. The PVDF membrane was washed three times with a TBS-T buffer, and visualized and quantified using an ECL Western blotting detection reagent.

Here, antibodies specific to elastin, caspase-3, collagen, MKP-1, p-cPLA, p-p38, NF-κB(p65), CK2α, GAPDH, or β-actin were used in the Western blot.

[Experimental Method 8] RNA Interference

Small interfering RNA (siRNA; hereinafter referred to as “MKP-1 siRNA”) against RNA encoding an MKP-1 protein, and control siRNA strands were purchased from SANTA CRUZ (USA). Then, siRNA was delivered to the mice using an in vivo-jet polyethylene imine (PEI, Polyplus-transfection) according to the instructions provided by the manufacturer. Specifically, 200 μL of a mixed solution obtained by adding MKP-1 siRNA to a 5% glucose solution was reacted at room temperature for 20 minutes. Thereafter, the reaction solution was injected into the tail vein of each mouse at 24 hours before sacrificing the mice. An effect of the MKP-1 siRNA interference was determined by measuring an expression level of the MKP-1 protein in the lung tissue through Western blot analysis.

[Experimental Method 9] Statistical Processing

All the experiments described in the Experimental Methods were performed at least three times, and 3 to 5 mice were used per group. Statistical analysis data was expressed as mean standard deviation, and statistical comparison was performed using a one-way ANOVA and a Fisher test. A significant difference between respective groups was determined using an unpaired Student's t-test, and a significant level of P value was set to less than 0.05.

[Experiment Result 1] Confirmation of Inhibitory Effect on Inflammatory Cells Introduced into Airway in Asthma

Preparation Example 3 (α,δ-NA-L-G) or α,δ N-acetyl-D-glutamine (α,δ-NA-D-G) was orally administered to the asthma-induced mice of Experimental Method 1 daily from the experimental starting date at a concentration of 50, 100, or 200 μg/kg/day, and the cell numbers of neutrophils and eosinophils introduced into the airway through the method of Experimental Method 3 were measured. The results are shown in A and B of FIG. 3. In this case, the cell numbers were measured at 10 hours (neutrophils) or 48 hours (eosinophils) after a secondary challenge.

Here, the α,δ N-acetyl-D-glutamine is represented by the following Formula 3, as follows:

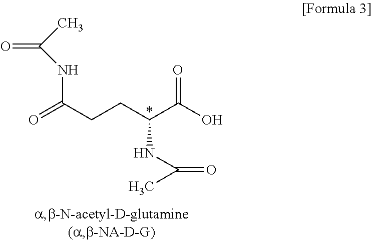 [SMILES: CC(=O)NC(=O)CC[C@@H](NC(C)=O)C(=O)O]

As shown in A of FIG. 3, the inflow of the neutrophils and eosinophils into the airway was very effectively inhibited when Preparation Example 3 was injected into the mice at a concentration of 50 to 200 μg/kg/day. In particular, Preparation Example 3 very effectively inhibited the neutrophils from a concentration of 50 μg/kg/day, compared to the eosinophils.

However, as shown in B of FIG. 3, when the α,δ N-acetyl-D-glutamine was administered, the inflow of the neutrophils and eosinophils into the airway was not inhibited regardless of the concentration.

Based on the results, it can be seen that the Preparation Example compound of the present invention very effectively inhibited the inflow of the neutrophils and eosinophils into the airway to treat asthma. Also, it can be seen that when the Preparation Example compound is in a “D” form, the inflow of the neutrophils and eosinophils into the airway was not inhibited.

[Experiment Result 2] Confirmation of Inhibitory Effect on Expression Level of Cytokines Present in Airway in Asthma

Preparation Example 3 (α,δ-NA-L-G) or α,δ N-acetyl-D-glutamine was orally administered to the asthma-induced mice of Experimental Method 1 at a concentration of 100, 200, 400 or 800 μg/kg/day 30 minutes before a secondary airway challenge, and levels of IL-4, IL-5, and IL-13 were measured through the method described above in Experimental Method 4 24 hours after the secondary challenge. The results are shown in A and B of FIG. 4.

As shown in A of FIG. 4, the levels of IL-4, IL-5, and IL-13 were remarkably inhibited by the administration of Preparation Example 3 at a concentration of 200 μg/kg/day.

However, as shown in B of FIG. 4, the administration of α,δ N-acetyl-D-glutamine did not inhibit the levels of the cytokines regardless of the concentration of α,δ N-acetyl-D-glutamine.

Based on the results, it can be seen that, when the Preparation Example compound of the present invention was orally administered, the Preparation Example compound remarkably inhibited the levels of IL-4, IL-5 and IL-13 corresponding to the Th2 cytokines, thereby very effectively inhibiting airway inflammation through eosinophils, the bronchial hypersensitivity reaction, and the production of IgE antibodies. In addition, it can be seen that when the Preparation Example compound is in a “D” form, the inflammatory cytokines were not inhibited.

[Experiment Result 3] Confirmation of Inhibitory Effect on Bronchial Hypersensitivity Reaction in Asthma

Preparation Example 3 (α,δ-NA-L-G) or α,δ N-acetyl-D-glutamine was orally administered to the asthma-induced mice of Experimental Method 1 at a concentration of 200 or 400 μg/kg/day 30 minutes before a secondary airway challenge, and the bronchial hypersensitivity reaction was measured 48 hours after the secondary challenge using the method described in Experimental Method 5. The results are shown in A and B of FIG. 5.

As shown in A of FIG. 5, it can be seen that when Preparation Example 3 was orally administered at a concentration of 200 or 400 μg/kg/day, Preparation Example 3 remarkably inhibited the Rrs value even at the increased concentration of methacholine.

However, as shown in B of FIG. 5, it was confirmed that when α,δ N-acetyl-D-glutamine was administered, the α,δ N-acetyl-D-glutamine did not exhibit an inhibitory effect on the Rrs value, which was observed in Preparation Example 3.

Based on the results, it can be seen that when the Preparation Example compound according to the present invention was orally administered, the Preparation Example compound very effectively inhibited the bronchial hypersensitivity reaction in asthma. Also, it can be seen that when the Preparation Example compound in a “D” form, the bronchial hypersensitivity reaction was not inhibited.

[Experiment Result 4] Confirmation of Inhibitory Effect on Inflammation of Lung Tissue in Asthma

Preparation Example 3 (α,δ-NA-L-G) was orally administered to the asthma-induced mice of Experimental Method 1 at a concentration of 200 or 400 μg/kg/day 30 minutes before a secondary challenge, and the lung tissue was stained using the method described above in Experimental Method 6 48 hours after the secondary challenge, and the inflammation score was measured. The results are shown in A and B of FIG. 6.

As shown in A and B of FIG. 6, a number of inflammatory cells were gathered around the airway in the group (OVA) to which ovalbumin was administered for the secondary challenge, the bronchus was narrowed, and blood vessels were formed around the bronchus. Thus, the inflammation score was 3. However, the degree of inflammation was remarkably inhibited in the group (OVA+ α,δ-NA-L-G (200 μg/kg/day)) to which Preparation Example 3 was administered at 200 μg/kg/day and the group (OVA+ α,δ-NA-L-G (400 μg/kg/day)) to which Preparation Example 3 was administered at 400 μg/kg/day. Thus, the inflammation score was reduced to 2 or 1, respectively.

Based on the results, it can be seen that when the Preparation Example compound according to the present invention was orally administered, the Preparation Example compound very effectively inhibited the inflammation caused in the lung tissue in asthma.

[Experiment Result 5] Confirmation of Cellular Signaling Associated with Inhibition of Asthmatic Response

[5-1] Confirmation of Expression and Activation Levels of MKP-1 Protein, p38 Protein, and cPLA2 Protein

After the secondary challenge (ovalbumin injection) was performed according to the method described above in Experimental Method 1, the levels of the MKP-1, p-cPLA, p-p38, and GAPDH proteins were measured at 10 minutes and 30 minutes according to the method described above in Experimental Method 7. The results are shown in FIG. 7. Here, Preparation Example 3 was orally administered daily at a concentration of 200, 500, or 1,000 μg/kg/day.

As shown in FIG. 7, the level of the MKP-1 protein increased from 10 minutes after the secondary challenge in the lung tissue of the asthma-induced mice, and such an increase in the MKP-1 protein was more increased depending on the concentration of Preparation Example 3. Meanwhile, the levels of the p-cPLA and p-p38 proteins decreased depending on the concentration of Preparation Example 3.

Based on the results, it can be seen that the novel compound according to the present invention increased the level of the MKP-1 protein, and inhibited the phosphorylation of the cPLA and p38 proteins.

[5-2] Confirmation of Inhibitory Effect on Phosphorylation by MKP-1 Inhibition

To determine whether the inhibitory effect on the phosphorylation of cPLA and p38 of Preparation Example 3 confirmed in Section [5-1] was dependent on MKP-1, the proteins were treated with MKP-1 siRNA under the same conditions as in Section [5-1], as described above in Experimental Method 8, and the levels of the proteins were measured according to the method described above in Experimental Method 7. The results are shown in FIG. 8. Here, as the control, the proteins were treated with the control siRNA strands instead of the MKP-1 siRNA, as described above in Experimental Method 8.

As shown in FIG. 8, when the MKP-1 protein was treated with siRNA, the level of the MKP-1 protein did not increase even when Preparation Example 3 was administered, and the phosphorylation levels of cPLA2 and p38 increased.

Based on the results, it can be seen that the novel compound according to the present invention increased the expression level of the MKP-1 protein, and also very effectively inhibited the phosphorylation of p38 and cPLA2.

[5-3] Confirmation of Inhibition of MKP-1 Protein-Dependent CK2/NF-κB Activation

It was confirmed whether the novel compound of the present invention could inhibit an inflammation-induced phenomenon in the bronchi by means of a mechanism in which a CK2α protein phosphorylates and activates an NK-κB protein by inhibiting the phosphorylation of p38 by the MKP-1 protein.

Specifically, the secondary challenge (ovalbumin injection) was performed according to the method described above in Experimental Method 1, and the lungs were extracted after an hour, and then measured according to the method described above in Experimental Method 7. The results are shown in A of FIG. 9. Also, the level of TNF-α corresponding to an NK-κB-dependent cytokine was measured by ELISA according to the method described above in Experimental Method 4. The results are shown in B of FIG. 9. Here, Preparation Example 3 was orally administered daily at a concentration of 400 μg/kg/day.

As shown in A and B of FIG. 9, the level of the CK2α protein increased through the ovalbumin injection, and accordingly, the levels of the NF-κB protein and TNF-α also remarkably increased (second column from the left of A and B of FIG. 9). However, when Preparation Example 3 was administered, the level of the CK2α protein decreased, and accordingly, the levels of NF-κB and TNF-α decreased (fifth column from the left of A and B of FIG. 9). As such, the effect of Preparation Example 3 for reducing the levels of CK2α, NF-κB, and TNF-α was not observed when the proteins were treated with siRNA specific to MKP-1.

Based on the results, it can be seen that the novel compound according to the present invention inhibited the expression level of the CK2α protein by means of the MKP-1 protein, and thus very effectively inhibited the levels of NF-κB and TNF-α, thereby very effectively treating a respiratory disease.

[5-4] Confirmation of Hierarchical Relationship Between CK2α Protein and p38

The secondary challenge (ovalbumin injection) was performed according to the method described above in Experimental Method 1, and the lungs were extracted after 1 or 2 hours, and then measured according to the method described above in Experimental Method 7. The results are shown in FIG. 10. Here, SB202190, which is a p38 inhibitor, was administered daily.

As shown in FIG. 10, when the p38 inhibitor SB202190 was administered, the level of the CK2α protein decreased 1 or 2 hours after the secondary challenge.

Based on the results, it can be seen that the CK2α protein is a protein that is present upstream of p38.

[5-5] Confirmation of NF-κB Activation Inhibitory Effect by Inhibition of CK2α Protein

The secondary challenge (ovalbumin injection) was performed according to the method described above in Experimental Method 1, and the lungs were extracted after 30 or 60 minutes, and then measured according to the method described above in Experimental Method 7. The results are shown in A and B of FIG. 11. Also, the level of TNF-α corresponding to an NK-κB-dependent cytokine was measured by ELISA according to the method described above in Experimental Method 4. The results are shown in B of FIG. 11. Here, TBBt, which is an inhibitor of the CK2α protein, was administered daily.

As shown in A and B of FIG. 11, when the inhibitor of the CK2α protein was administered, the level of the NF-κB protein decreased at 30 and 60 minutes. In addition, the level of the presence of the TNF-α protein regulated by NF-κB also decreased.

Based on the results, it can be seen that the novel compound of the present invention induced the activation of the MKP-1 protein to block a cellular signaling pathway proceeding in the order of p38/CK2α/NF-κB, thereby very effectively inhibiting an inflammatory response occurring in a respiratory disease.

[5-6] Confirmation of Loss of Asthma Inhibitory Effect by Inhibition of MKP-1 Protein

siRNA specific to MKP-1 was treated according to the method described above in Section [5-2], and the number of inflammatory cells, expression levels of the cytokines, a degree of hypersensitivity reaction in the bronchus, and a degree of inflammation in the lung tissue were determined in the same manner as described in Experiment Results 1 to 3. The results are shown in FIGS. 12 to 15.

As shown in FIGS. 12 to 15, when Preparation Example 3 was administered, the cell numbers of neutrophils and eosinophils decreased, the expression levels of the cytokines (IL-4, IL-5, and IL-13) decreased, and the degree of inflammation in the lung tissue was inhibited. However, when Preparation Example 3 and siRNA specific to MKP-1 were treated, the decreased cell numbers of the neutrophils and eosinophils increased, the expression levels of the cytokines also increased, and an inflammation-relieving effect also disappeared.

Based on the results, it can be seen that the asthma inhibitory effect of the novel compound according to the present invention was induced by increasing the expression level of the MKP-1 protein.

In summary, as shown in FIG. 16, the novel compound according to the present invention increased the expression level of the MKP-1 protein to inhibit the activities of the p38 and PLA2 proteins, and thus inhibited the expression levels of the cytokines (TNF-α, IL-4, IL-5 and IL-13) expressed through a cellular signaling pathway proceeding in the order of p38/CK2α/NF-κB and inhibited the inflammatory response induced by eicosanoids, thereby very effectively inhibiting the inflammatory response occurring in the bronchus.

[Experiment Result 6] Confirmation of Effect of Inhibiting Expression Levels of Proteins Increased in Chronic Obstructive Pulmonary Disease

Western blot analysis was performed according to the method described above in Experimental Method 7 for the group (30) to which Preparation Example 3 (α,δ-NA-L-G) was orally administered at 250 μg/kg/day for the last 15 days while inducing a chronic obstructive pulmonary disease (COPD) for 30 days in the mice according to the method described above in Experimental Method 1; or the group (60) to which Preparation Example 3 was orally administered at 250 μg/kg/day for the last 30 days while inducing the COPD for 60 days, and the levels of elastin, collagen, and caspase-3 proteins were measured. The, results are shown in FIGS. 17 and 18.

As shown in FIGS. 17 and 18, when COPD was induced for 30 and 60 days, the levels of the elastin, collagen, and caspase-3 proteins increased (COPD), but the levels of the proteins decreased to that of the negative control (saline) when Preparation Example 3 was administered (COPD+α,δ-NA-L-G).

Based on the results, it can be seen that when the novel compound according to the present invention was orally administered, the novel compound remarkably inhibited the expression levels of the elastin, collagen and caspase-3 proteins increased in COPD, and thus is able to be used to very effectively treat COPD as well as asthma.

[Experiment Result 7] Confirmation of Inhibitory Effect of Novel Compound on Inflammatory Response in Respiratory System

The number of inflammatory cells, expression levels of cytokines, and a degree of hypersensitivity reaction in the bronchus were determined according to the method described above in Experiment Result 1 to 3. The results are shown in FIGS. 19 to 21. Here, in order to determine whether the other compounds prepared according to the present invention have equivalent effects, Preparation Example 2 (α,δ-NA-L-G-Bn), Preparation Example 4 (α,δ-NA-L-G-Et) or Preparation Example 6 (α,δ-NA-L-G-Me) was orally administered daily instead of Preparation Example 3.

As shown in FIGS. 19 to 21, Preparation Examples 2, 4, and 6 also decreased the cell numbers of neutrophils and eosinophils like Preparation Example 3, decreased the expression levels of the cytokines (IL-4, IL-5, and IL-13), and also inhibited the bronchial hypersensitivity reaction occurring in the lung tissue.

Based on the results, it can be seen that Preparation Examples sharing the same backbone as the core very effectively inhibited the inflammatory response in the respiratory system similarly to Preparation Example 3.

The above description of the present invention has been given by way of illustration only. Therefore, those skilled in the art to which the present invention pertains will appreciate that the present invention can be embodied in other specific forms without changing the technical spirit or essential features of the present invention. Therefore, it should be understood that the embodiments described above are for the purpose of illustration only, and not intended to be limiting in all respects.

### INDUSTRIAL APPLICABILITY

The novel compound according to the present invention is effective in inducing the activation of MKP-1 protein to block a cellular signaling pathway proceeding in the order of p38/CK2α/NF-κB, thereby very effectively inhibiting an inflammatory response occurring in a respiratory disease. Therefore, the novel compound according to the present invention is industrially applicable because a respiratory disease can be prevented, alleviated, or treated by orally administering the novel compound according to the present invention.

1. A method of treating or alleviating a respiratory disease, comprising administering to a subject in need thereof a composition comprising an effective amount of a compound selected from a compound represented by the following Formula 1, a pharmaceutically acceptable salt, a hydrate, and a solvate thereof: 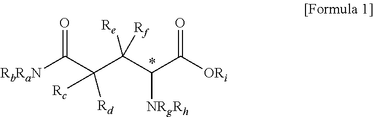
wherein:
Rₐ and R_{b} are each independently H or —C(═O)—Rⱼ;
R_{c} to R_{f} are each independently H or a C1-C6 alkyl group;
R_{g} and Rₕ are each independently H, a C1-C6 alkyl group, —C(═O)—Rₖ, or —C(═O)—O-L₂-Rₗ;
Rᵢ is H or a C1-C6 alkyl group;
Rⱼ and Rₖ are each independently a C1-C6 alkyl group;
Rₗ is a C1-C6 alkyl group, a C6-C12 aryl group, or a non-aromatic condensed polycyclic group having 5 to 20 nuclear atoms;
L₂ is a direct bond or a C1-C6 alkylene group;
the alkyl group of Rᵢ is substituted or unsubstituted with one or more C6-C12 aryl groups, which are the same or different from each other when substituted with a plurality of substituents; and
* represents a chiral center.

2. The method of claim 1, wherein the compound is in an L form based on the chiral center represented by *.

3. The method of claim 1, wherein the compound represented by Formula 1 is represented by the following Formula 2: 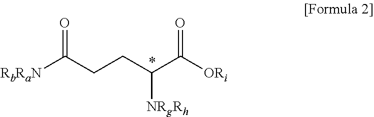
wherein:
Rₐ and R_{b} are each independently H or —C(═O)—Rⱼ;
R_{g} and Rₕ are each independently H, a C1-C6 alkyl group, —C(═O)—Rₖ, or —C(═O)—O-L₂-Rₗ;
Rᵢ is H or a C1-C6 alkyl group;
Rⱼ and Rₖ are each independently a C1-C6 alkyl group;
Rₗ is a C1-C6 alkyl group, a C6-C12 aryl group, or a non-aromatic condensed polycyclic group having 5 to 20 nuclear atoms;
L₂ is a direct bond or a C1-C6 alkylene group;
the alkyl group of Rᵢ is substituted or unsubstituted with one or more C6-C12 aryl groups, which are the same or different from each other when substituted with a plurality of substituents; and
* represents a chiral center.

4. The method of claim 1, wherein the compound is one or more selected from the group consisting of the following compounds:

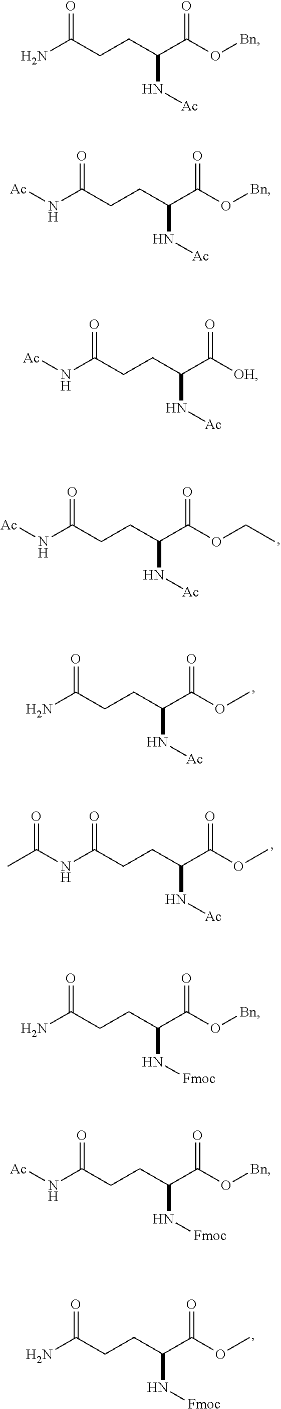

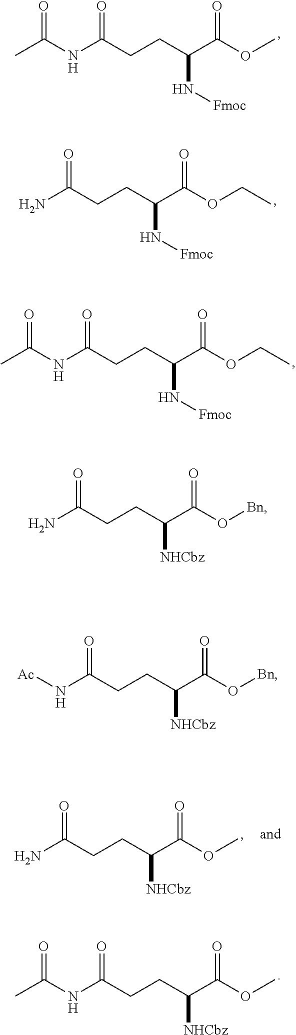

5. The method of claim 1, wherein the respiratory disease is one or more selected from the group consisting of a cold accompanied by coughing or phlegm, influenza, asthma, a chronic obstructive pulmonary disease, bronchial adenoma, a solitary pulmonary nodule, pulmonary tuberculosis, empyema, a pulmonary abscess, and pulmonary histiocytosis.

6. The method of claim 5, wherein the chronic obstructive pulmonary disease has one or more symptoms of chronic bronchitis or pulmonary emphysema.

7. The method of claim 1, wherein the composition is a pharmaceutical composition or food composition.

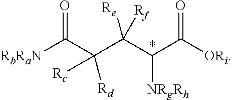

8. A composition for preventing, treating, or alleviating a respiratory disease, comprising as an active ingredient a compound selected from a compound represented by the following Formula 1, a pharmaceutically acceptable salt, a hydrate, and a solvate thereof: 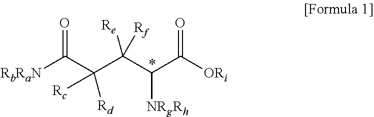
wherein:
Rₐ and R_{b} are each independently H or —C(═O)—Rᵢ;
R_{c} to R_{f} are each independently H or a C1-C6 alkyl group;
R_{g} and Rₕ are each independently H, a C1-C6 alkyl group, —C(═O)—Rₖ, or —C(═O)—O-L₂-Rₗ;
Rᵢ is H or a C1-C6 alkyl group;
Rᵢ and Rₖ are each independently a C1-C6 alkyl group;
Rₗ is a C1-C6 alkyl group, a C6-C12 aryl group, or a non-aromatic condensed polycyclic group having 5 to 20 nuclear atoms;
L₂ is a direct bond or a C1-C6 alkylene group;
the alkyl group of Rᵢ is substituted or unsubstituted with one or more C6-C12 aryl groups, which are the same or different from each other when substituted with a plurality of substituents; and
* represents a chiral center.

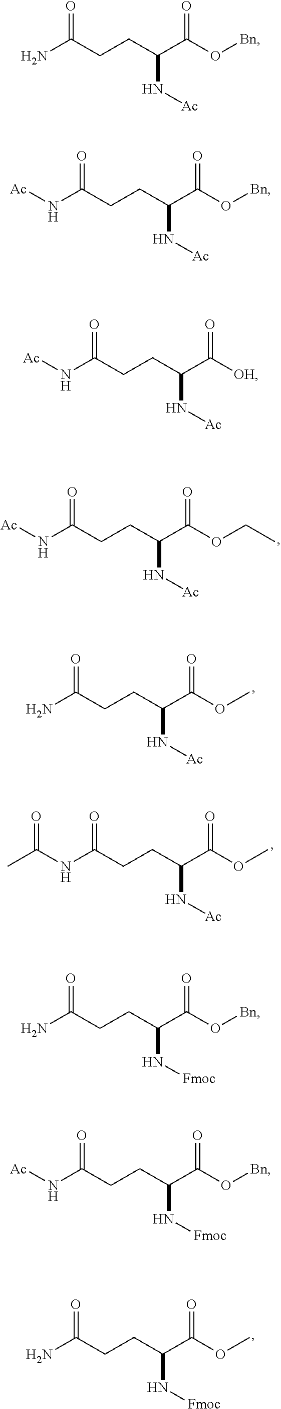

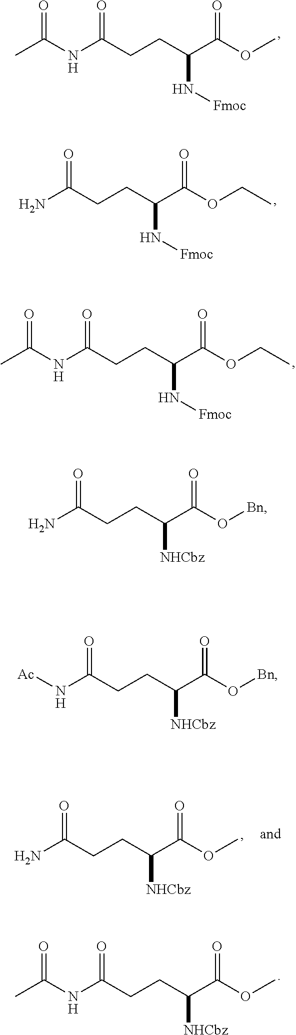

9. The composition of claim 8, wherein the composition is a pharmaceutical composition or food composition.

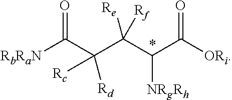

10.-11. (canceled)
